(19) United States
(12) Patent Application Publication
(10) Pub. No.: US 2016/0058937 A1
(43) Pub. Date: Mar. 3, 2016

(54) BLOOD CLEANSING AND APPARATUS & METHOD

(71) Applicants: Angelo Gaitas, Miami, FL (US); Gwangseong Kim, Miami, FL (US)

(72) Inventors: Angelo Gaitas, Miami, FL (US); Gwangseong Kim, Miami, FL (US)

(73) Assignee: Angelo Gaitas, Miami, FL (US)

(21) Appl. No.: 14/936,112

(22) Filed: Nov. 9, 2015

Related U.S. Application Data
(63) Continuation-in-part of application No. 14/567,784, filed on Dec. 11, 2014.
Application No. 14/567,784 is a continuation-in-part of application No. 14/564,042, filed on Dec. 8, 2014.
Application No. 14/564,042 is a continuation-in-part of application No. 14/482,270, filed on Sep. 10, 2014.
(60) Provisional application No. 61/900,070, filed on Nov. 5, 2013.

(51) Int. Cl. A61M 1/36

(52) U.S. Cl. CPC A61M 1/3683; A61M 1/362; A61M 1/3679

(57) ABSTRACT

The present invention relates to removing disease causing agent such as pathogens from the blood of a patient. Specifically, the invention relates to using coating materials to trap disease causing agent that is desired to be removed from the blood of a patient. It also related to using lights of specific wavelength to inactivate pathogens. The light is used to activate reactive oxygen species using a photo-sensitizer or directly kill the pathogen using light of wavelength between 100 nm and 450 nm.

### CROSS REFERENCE TO RELATED APPLICATIONS

This application is a continuation-in-part of U.S. Non-Provisional Utility patent application Ser. No. 14/567,784 filed Dec. 11, 2014, and entitled “Blood Cleansing System & Method”, which is a continuation-in-part of U.S. Non-Provisional Utility patent application Ser. No. 14/564,042 filed Dec. 8, 2014, and entitled “Blood Cleansing System”, which is a continuation-in-part of U.S. Non-Provisional Utility patent application Ser. No. 14/482,270 filed Sep. 10, 2014, and entitled “Blood Cleansing System”, each of which claims the benefit of U.S. Provisional Patent Application No. 61/900,070 filed Nov. 5, 2013 and entitled “Blood Cleansing System,” the entire disclosures of each and all of the above mentioned references are hereby incorporated herein by reference.

### GOVERNMENT SUPPORT

This invention was made with government support under U.S. Public Health Service Grant No. GM084520 from the National Institutes of Health. The Government has certain rights in the invention.

### FIELD OF THE INVENTION

The present invention relates to removing disease causing agent from the blood of a patient. Specifically, the invention relates to using coating materials to trap disease causing agent that is desired to be removed from the blood of a patient.

### BACKGROUND OF THE INVENTION

Many diseases, as well as other harmful particles and biological molecules, are carried by the blood. While there are certain methods directed towards filtering toxins from the blood, existing systems and methods do not target specific particles for removal from the blood. In general, for cell capturing, a cell surface marker is targeted, such as a protein or receptor on the membrane, using a coating material (such as antibody or aptamer) linked to a cleansing chamber's surface. However, there are no existing methods that utilize the previously mentioned capture technique to target and remove particles from the blood.

Microorganism infections in the bloodstream, especially those caused by drug-resistant strains, are a major cause of death and afflict millions of people and animals worldwide. For instance, sepsis, which is frequently acquired in hospitals, causes septic shock and death with a very high mortality rate. Additionally, Methicillin-resistant Staphylococcus aureus (MRSA)) kills thousands of Americans, with annual treatment costs in the billions of dollars. Furthermore, parasitic protozoans such as malaria (especially artemisinin resistant Plasmodium strains) pose a serious threat for global health.

Therefore there is a need in the art for a system and method to remove unwanted particles, cells, and bio-molecules from blood by targeting specific particles. These and other features and advantages of the present invention will be explained and will become obvious to one skilled in the art through the summary of the invention that follows.

### SUMMARY OF THE INVENTION

Accordingly, it is an object of the present invention to provide a method for removing disease causing agent from the blood of a patient. In some embodiments this invention is used to reduce metastatic cancer. In cancer metastasis cells from a primary tumor become circulating tumor cells (CTCs) and then adhere to other organs to create a metastasis. This invention discloses a method and an apparatus to remove disease causing agent. According to an embodiment of the present invention, the disease causing agent is one or more disease causing agents selected from a group of disease causing agents comprising: cancer stem cells, metastatic cancer cells, cancer cells, circulating tumor cells, viruses, microorganisms, bacteria, peptides, beta amyloid (Amyloid beta, Aβ, Abeta), proteins, enzymes, toxins, diseased cells, cells, fungi, pathogens, materials, Carbapenem-resistant Enterobacteriacea, CRE bacteria, Ebola, Malaria, cholesterol, glucose, parasitic protozoans, Klebsiella pneumoniae Carbapenemase (KPC)-Producing Bacteria, Alzheimer's causing material, diseased cells, septic shock and sepsis infection causing organisms, lactate, stem cell-like cancer cells, biomolecules, HIV virus, Methicillin-resistant Staphylococcus aureus, bacteremia, toxic materials, mesenchymal tumor cells, herpes, herpes viruses, parasites, cytokines, and other deleterious material that is desired to be removed from blood.

According to an embodiment of the present invention, a patient's blood is pumped and flowed through a cleansing chamber that contains one or more of the following: a filter or filters or a cleansing chamber with pillars (or micropillars), micro-posts, tube or tubes, well(s) with a microfluidic reaction chamber (made of a spiraling microfluidic tube), microspheres (beads or microbeads) or spheres, or any combination thereof. Coating materials have been pre-coated on the cleansing chamber or on parts of the cleansing chamber such as the microspheres. Alternatively, the cleansing chamber may include a mechanism for size separation. In some embodiments, the cleansing chamber may include a semi-permeable membrane. In a preferred embodiment, as blood flows through the cleansing chamber, undesired substances are trapped (for example CTCs) while red blood cells and desired substances are re-circulated back into the patient. The process can be repeated several times. In some embodiments, the trapped substances are further analyzed to examine and study disease progression.

According to an embodiment of the present invention, a method for removing disease causing agent from blood includes the steps of: pumping blood from a patient into a cleansing chamber; flowing said blood through said cleansing chamber to expose said blood to a coating material; capturing disease causing agent, wherein said coating material targets and binds to said disease causing agent; removing said disease causing agent from said blood; and returning said blood to said patient.

According to an embodiment of the present invention, the blood is pumped to said cleansing chamber until said cleansing chamber is full thereby allowing said coating material to capture said disease causing agent.

According to an embodiment of the present invention, the coating material is one or more coating materials selected from a group of coating materials comprising antibodies, peptides, proteins, aptamers, nucleic acid, RNA, DNA, organic materials, magnetic particles, TNF-related apoptosis-inducing ligands (TRAIL), ligands, apoptosis inducing substances, death receptors binding substances, tumor necrosis factors, adhesion receptors, E-selectin, cytokines, chemotherapy agents, biological binders. According to an embodiment of the present invention, the cleansing chamber is coated with a coating material, wherein the coating material is selected from the group of coating materials comprising amoxicillin, molecules that adhere to penicillin binding proteins, molecules that adhere to alpha-gal, clavulanic acid, microorganism killing compounds, β-lactam antibiotics, molecules such as antibodies and peptides that target microorganism's cell walls, molecules that target FtsZ protein, synthetic antibacterials, PC190723, molecules that inhibit FtsZ, substances that induce apoptosis, substances that bind to certain death receptors, tumor necrosis factors (or the TNF family), adhesion receptors, photosensitizer-linked antibodies, photosensitizers for photodynamic therapy, malarial protein VAR2CSA, rVAR2-diphtheria toxin fusion, rVAR2-hemiasterlin conjugate, rVAR2, Nilotinib, Paclitaxel, E-selectin, and cytokines. One of ordinary skill in the art would appreciate there are numerous coatings that might be used and embodiments of the present invention are contemplated for use with any such coating. In some cases the coating material is also referred to as binding material, in this disclosure “antibody” is used as an example, however this particular coating material can be replaced with any other binding agent in the cleansing chamber or as a conjugate material to the photosensitizer. In another embodiment, this method can be applied to other conditions requiring blood cleansing including, but not limited to sepsis, poisoning, leukemia, and cholesterols.

According to an embodiment of the present invention, the method further includes the step of analyzing said disease causing agent that has been captured by said coating material.

According to an embodiment of the present invention, the method further includes the step of counting the amount of said disease causing agent trapped in said cleansing chamber.

According to an embodiment of the present invention, the cleansing chamber is comprised of an inlet, an outlet, and a cleaning mechanism for removing said disease causing agent.

According to an embodiment of the present invention, an inner surface of said cleansing chamber is coated with said coating material.

According to an embodiment of the present invention, the cleansing mechanism is comprised of a plurality of spheres, each of has an outer surface that is coated with said coating material.

According to an embodiment of the present invention, the cleansing mechanism is comprised of a plurality of pillars, each of which is coated with said coating material.

According to an embodiment of the present invention, the cleansing mechanism is comprised or one or more tubes, each of which has an inner surface that is coated with said coating material.

According to an embodiment of the present invention, the cleansing mechanism is further comprised of a nanorough surface.

According to an embodiment of the present invention, the cleansing mechanism is further comprised of a microrough surface.

According to an embodiment of the present invention, a method for removing disease causing agent from blood, said method comprising the steps: of attaching a photosensitizer with a binding agent to generate a conjugate material; injecting the conjugate material into a patient such that the conjugate material binds to a disease causing agent; circulating blood through a cleansing chamber such as an extracorporeal transparent tube; illuminating said tube with light to activate said photosensitizer, wherein the activation of said photosensitizer releases reactive oxygens capable of causing cell death upon contact with the oxygen. In some embodiments the conjugate photosensitizer-binding material is introduced via a port connected to the tube connected to the outlet of the cleansing chamber, with the port being used to introduce a photosensitizer to the patient. A conjugate material is a material that combines a photosensitizer and a binding agent. A cleansing chamber, such as an extracorporeal transparent tube, is a hollow cylindrical or any other shape transparent (allowing light of at least a specific wavelength to go though the cleansing chamber) cleansing chamber that liquids can be flowed through.

According to embodiments of the current method, the cleansing chamber is selected from a group of cleansing chamber comprising PDMS, organic material, glass, quartz, plastic, polymer, metallic and silicone cleansing chambers.

According to embodiments of the claimed method, the cleansing chamber is a extracorporeal transparent tube with inner diameter is selected from a group of inner diameters of 1.02 mm, 0.5 mm, 1 mm, 0.8 mm, 2 mm, 3 mm. According to another embodiment of the claimed method, the extracorporeal transparent tube has an inner diameter of less than 2 mm.

According to embodiments of the claimed method, the cleansing chamber is modified with one or more additional coating materials to capture said disease causing agent.

According to another embodiment of the claimed method, a series of cleansing chambers are used joined to each other, each cleansing chamber containing a different coating material to capture or kill disease causing agent.

According to embodiments of the claimed method, the coating material can be one or more of antibodies, protein, peptide or one or more of a material that binds to a disease causing agent. A coating material is a substance that binds to the disease causing agent.

According to embodiments of the claimed method, the photosensitizer is modified with a crosslinker to make it receptive to a binding agent.

According to embodiments of the claimed method, the light used to activate the photosensitizer is including, but not limited to UVA (320-400 nm), 470 nm, 537 nm, 630 nm, 625 nm, 660 nm, 780 nm or other wavelengths depending on the excitation maxima of given photosensitizers.

According to embodiments of the claimed method, the conjugate material is used as an imaging agent.

According to an embodiment of the present invention, an apparatus for removing disease causing agent from blood comprising: an inlet tube for flowing blood from a blood source; a pump connected to the tube; one or more cleansing chambers connected to the tube, wherein each of the cleansing chambers comprises an inlet, through which the blood flows in to the cleansing chamber, an outlet, through which the blood flows out of the cleansing chamber, an outlet, and an inner portion coated with a coating material; and an outlet tube connected to the outlet of the cleansing chamber which returns the blood to the blood source.

According to an embodiment of the present invention, each of the cleansing chamber is one or more cleansing chambers selected from a group of cleansing chambers comprising tube, parallelepiped, rectangular parallelepiped, and a cylinder.

According to an embodiment of the present invention, the coating material is one or more coating materials selected from a group of coating material comprising antibodies, adhesion molecules, and pathogen killing molecules.

According to an embodiment of the present invention, the apparatus for removing disease causing agents from blood further comprises one or more light sources each of which illuminate at least one of the one or more cleansing chambers.

According to an embodiment of the present invention, the light sources generate light with one or more of wavelengths selected from a group of wavelengths comprising a wavelength centered at 207 nm, a wavelength centered at 415 nm, a wavelength centered at 400 nm, a wavelength centered at 405 nm, a wavelength centered at 200 nm, a wavelength between 100 nm and 210 nm, a wavelength between 100 nm and 400 nm, a wavelength between 380 nm and 450 nm, 660 nm, a wavelength between 650 nm and 700 nm, a wavelength between 700 nm and 900 nm, a wavelength longer than 400 nm, and a wavelength that activates a photosensitizer.

According to an embodiment of the present invention, the blood source is a patient receiving treatment.

According to an embodiment of the present invention, the apparatus for removing disease causing agents from blood further comprises a port connected to the outlet tube, wherein the port is used to introduce a photosensitizer to the blood source.

According to an embodiment of the present invention, an apparatus for removing disease causing agent from blood of a patient comprising: an inlet tube for flowing blood from a blood source; a pump connected to the tube; one or more cleansing chambers connected to the tube, wherein each of the cleansing chambers comprises an inlet, through which the blood flows in to the cleansing chamber, an outlet, through which the blood flows out of the cleansing chamber, and an outlet; one or more light sources each of which illuminate at least one of the one or more cleansing chambers; and an outlet tube connected to the outlet of the cleansing chamber which returns the blood to the blood source.

According to an embodiment of the present invention, the apparatus for removing disease causing agents from blood further comprises a coating material on an inner part of one or more of the cleansing chambers, wherein the coating material is one or more coating materials selected from a group of coating material comprising antibodies, adhesion molecules, and pathogen killing molecules.

According to an embodiment of the present invention, a method for removing disease causing agent from blood comprising the steps of: flowing blood from a patient through a tube to a first cleansing chamber; illuminating the blood with light from a light source that generates light with wavelength one or more of wavelengths selected from a group of wavelengths comprising a wavelength centered at 207 nm, a wavelength centered at 415 nm, a wavelength centered at 400 nm, a wavelength centered at 405 nm, a wavelength centered at 200 nm, a wavelength between 100 nm and 210 nm, a wavelength between 100 nm and 400 nm, a wavelength between 380 nm and 450 nm, 660 nm, a wavelength between 650 nm and 700 nm, a wavelength between 700 nm and 900 nm, a wavelength longer than 400 nm, and a wavelength that activates a photosensitizer; flowing blood through a second cleansing chamber configured with a coating material on an inner part of the second cleansing chamber, wherein the coating material is one or more coating materials selected from a group of coating material comprising antibodies, adhesion molecules, and pathogen killing molecules; and flowing blood out of the second cleansing chamber into a tube.

According to an embodiment of the present invention, a method for removing disease causing agent from blood further comprising the steps of: injecting a photosensitizer into the patient, wherein the photosensitizer attaches to the disease causing agent; and illuminating the first cleansing chamber with light from the light source to activate the photosensitizer, wherein the activation of the photosensitizer generates reactive oxygen species that cause cell death to the disease causing agent upon contact with the reactive oxygen species.

16. The method of claim 13, wherein the second cleansing chamber is configured with one or more additional binding agents to capture said disease causing agent.

The foregoing summary of the present invention with the preferred embodiments should not be construed to limit the scope of the invention. It should be understood and obvious to one skilled in the art that the embodiments of the invention thus described may be further modified without departing from the spirit and scope of the invention.

### BRIEF DESCRIPTION OF THE DRAWINGS

FIG. 1 is an illustration of a patient's blood being pumped and flown through the cleansing chamber, after which the cleansed blood is injected/circulated back into the patient.

FIG. 2 is an illustration of a patient's blood being pumped and flown through the cleansing chamber, after which the cleansed blood is injected back into the patient.

FIG. 3 is an illustration a pressure monitor, a anticoagulant (such as heparin) pump, and an inflow pressure monitor in accordance with an embodiment of the present invention.

FIG. 4 is an illustration of blood flowing from the patient through a tube to a cleansing chamber with spheres that include a coating material.

FIG. 5 is an illustration of a cleansing chamber including pillars coated with coating material, in accordance with an embodiment of the present invention.

FIG. 6 is an illustration of a cleansing chamber composed of tube(s) coated with coating material, in accordance with an embodiment of the present invention.

FIG. 7 is an illustration of a cleansing chamber that uses filtering to separate wanted from unwanted material in the blood, in accordance with an embodiment of the present invention.

FIG. 8 is an illustration of a tube with captured material for removal, in accordance with an embodiment of the present invention.

FIG. 9 is an illustration of a light or radiation exposure unit included on the cleansing chamber to achieve photochemotherapy or radiotherapy.

FIG. 10 shows the steps of a tube coating process, in accordance with an embodiment of the present invention.

FIG. 11 shows the steps of a tube coating process, in accordance with an embodiment of the present invention.

FIG. 12 contains pictures of actual tubes with fluorescently labeled captured cells, in accordance with an embodiment of the present invention.

FIG. 13 shows the steps of a tube coating process, in accordance with an embodiment of the present invention.

FIG. 14 shows a schematic of the method of the claimed invention, in accordance with an embodiment of the present invention.

FIG. 15 illustrates the effect of light absorbing blood on photodynamic therapy.

FIG. 16 illustrates the effect of photodynamic therapy.

FIG. 17 illustrates a quantitative comparison of the effect of photodynamic therapy.

FIG. 18 is a conceptual diagram of an extracorporeal cleansing chamber where blood is circulated through the tube with peristaltic pumping.

FIG. 19 is a conceptual diagram illustrating the capture of pathogens by antibody coating material immobilized on a cleansing chamber wall.

FIG. 20 is a conceptual diagram of the various components of a blood cleansing apparatus.

FIG. 21 is an illustration of a cleansing chamber design, in accordance with an embodiment of the present invention.

FIG. 22 is an illustration of a cleansing chamber design, in accordance with an embodiment of the present invention.

FIG. 23 illustrates the various tubes and tube connectors of a cleansing chamber device, in accordance with embodiments of the present invention.

FIG. 24 is a conceptual diagram of a blood cleaning device of the present invention configured as a dialysis-like apparatus or part of a dialysis machine.

### DETAILED SPECIFICATION

The present invention relates to removing disease causing agent from the blood of a patient. Specifically, the invention relates to using coating materials to trap disease causing agent that is desired to be removed from the blood of a patient.

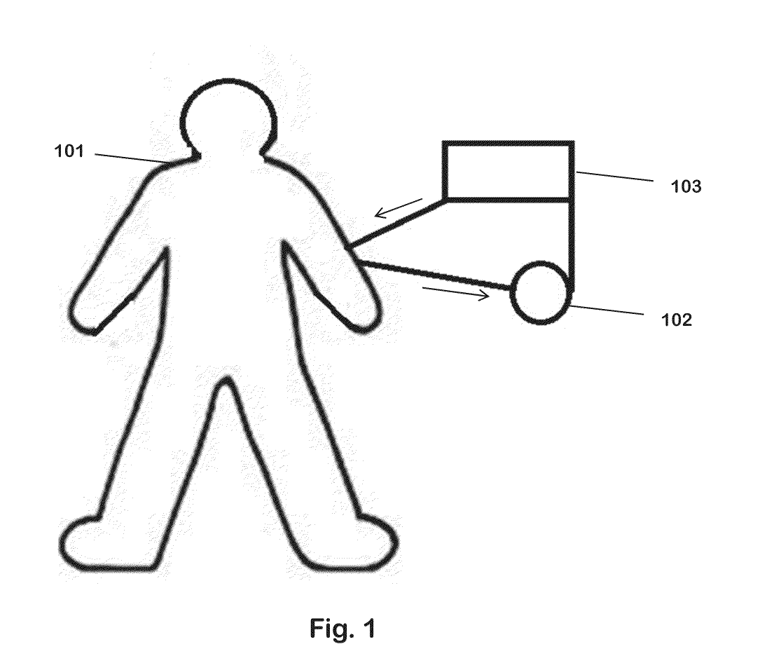

According to an embodiment of the present invention, the invention can utilize coating materials such as biological binders (i.e. antibodies) to trap microorganisms. In a preferred embodiment, the blood is decontaminated and returned to the body. The invention may utilize coating material in the form of biological binders, such as antibodies or peptides, to trap disease causing agent, such as a pathogen, a cell, a cancer cell, polymer, chemical compound, or folic acid. According to an embodiment of the present invention, as shown in FIG. 1, the patient's (101) blood is moved by a pump (102) and flowed through the cleansing chamber (103). After the cleansing process is complete, the patient's blood is injected back in the patient.

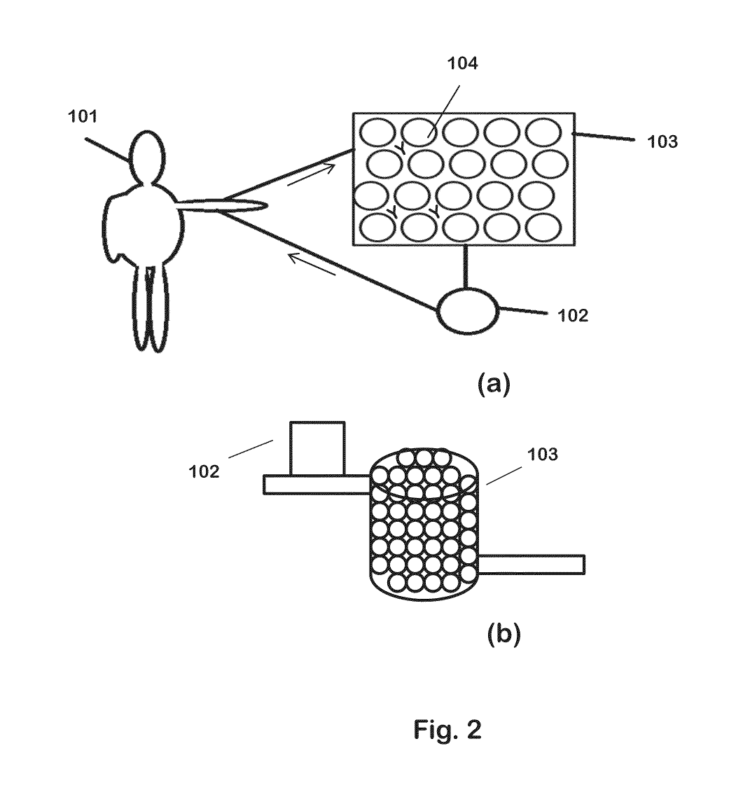

According to an embodiment of the present invention, as shown in FIG. 2 (a), the patient's (101) blood is moved by a pump (102) and flown through the cleansing chamber (103). After the cleansing process is complete, the patient's blood is injected back in the patient. In some embodiments, the cleansing chamber (103) contains spheres with specific coating materials, such as antibodies (104), that target and bind to the specific particles that are desired to be removed from the patient's blood. In some embodiments, shown in FIG. 2 (b), the cleansing chamber (103) is a column partially or entirely backed with beads, for instance a glass bead column. In the preferred embodiment, the glass tube may be configured in a variety of dimensions depending upon a particular application, with a preferred diameter of between 1 mm and 50 mm and a preferred height between 5 cm and 1 m. The beads may be pre-coated with coating material to trap and/or kill the disease causing agents. Gravity or a pump (102) may be used to push the blood through the apparatus. In the preferred embodiment, the beads are made of any suitable material including, but not limited to glass, silica gel, or other appropriate materials. The diameter of the glass beads can be configured in a variety of ranges including, but not limited to, 1 micron, 10 micron, 40-63 micron, 63-200 micron, 0.5 mm, and 1 mm.

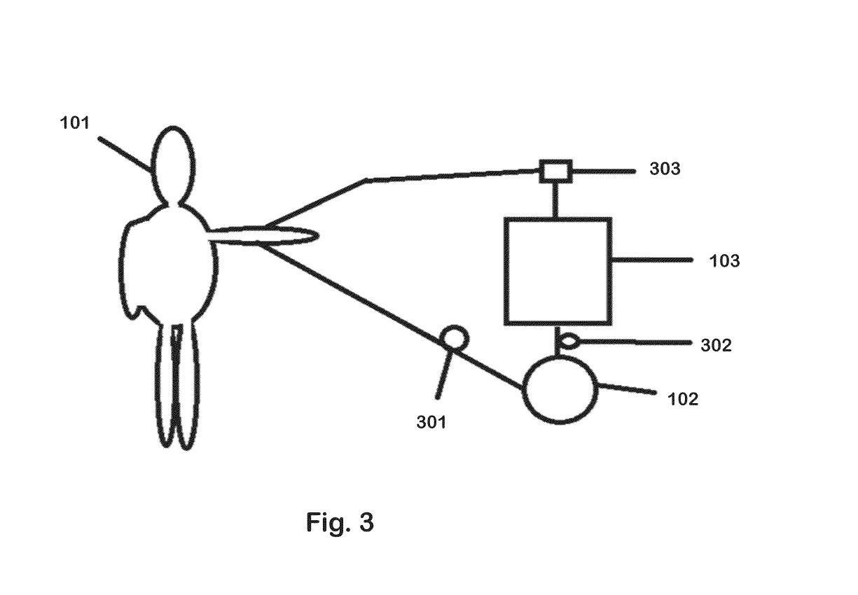

According to an embodiment of the present invention, as shown in FIG. 3, a pressure monitor (301) may be used to measure arterial pressure. In some embodiments, a anticoagulant (such as heparin) pump (302) and an inflow pressure monitor may also be included. In some embodiments, a venous pressure monitor and/or an air trap and air detector (303) are also included. Certain embodiments of the present invention may include fewer or additional components and the present invention may be used with any combination of the mentioned and additional components to achieve the desired functionality. One of ordinary skill in the art would appreciate that the cleansing chamber may be configured with any number of components based upon the desired functionality for the cleansing chamber, and embodiments of the present invention are contemplated for use with any such component.

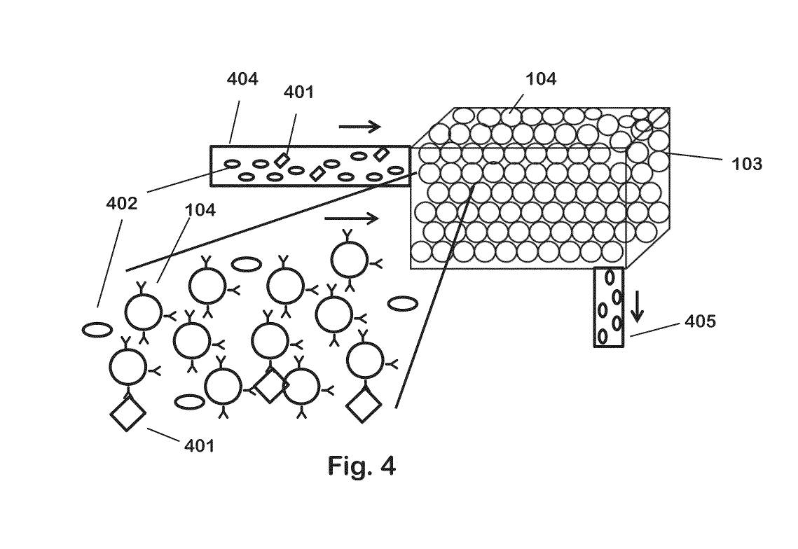

According to an embodiment of the present invention, as shown in FIG. 4, blood flows from the patient through a tube to the cleansing chamber (103). In the preferred embodiment, the cleansing chamber (103) includes spheres with coating material (104). In some embodiments, the coating materials are antibodies or aptamers specific to the cell surface marker of the cells that are being targeted for removal, such as circulating tumor cells (CTCs) (401). CTCs detach from both primary and metastatic lesions and attach to other areas on the body. As disease causing agent (401) such as CTCs flow through the cleansing chamber, (103) they are captured and removed (as shown in FIG. 4). The resulting output blood is clean of unwanted material and is returned to the body of the patient. In some embodiments, the surface of the cleansing chamber (103) or of the sphere (104) (or of the tube or of the pillar) is a nanorough surface that captures cells such as CTCs. A nanorough surface possesses nanometer scale roughness. A microrough surface possesses micrometer scale roughness. One of ordinary skill in the art would appreciate that the cleansing chamber could be used with any coating material, and embodiments of the present invention are contemplated for use to target and remove any cell type.

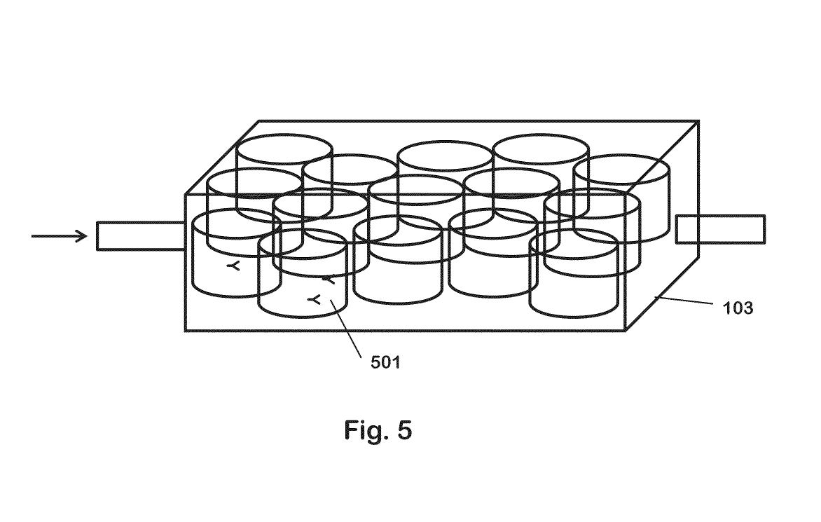

According to an embodiment of the present invention, in FIG. 5, the cleansing chamber (103) includes pillars (501) coated with coating material. In a preferred embodiment, the pillars are tightly positioned to increase the chances that the desired particles will collide and stick to the pillars. One of ordinary skill in the art would appreciate that there would be many useful patterns and arrangements that the pillars could be positioned in, and embodiments of the present invention are contemplated for use with any such arrangement.

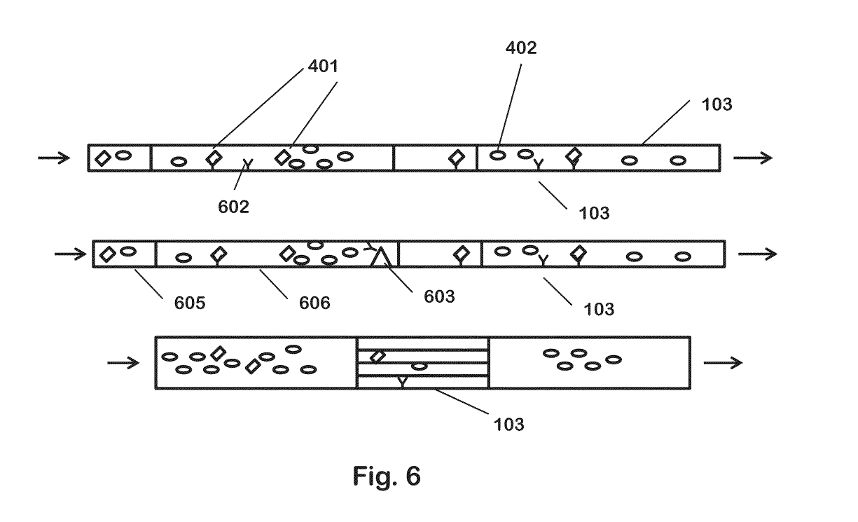

According to an embodiment of the present invention, as shown in FIG. 6, the cleansing chamber is composed of tubes (103), for example flexible tubes, coated with coating material (603) such as adhesion protein. In some embodiments the flexible tube includes a nanorough or microrough surface. In some embodiments, multiple tubes join together (for example 605 and 606), with each tube having different coating materials (602), such as different antibodies for separate diseases. In a preferred embodiment, this allows the cleansing chamber to target and remove multiple types of disease causing agents such as cell types from the blood. In a preferred embodiment, as blood flows out of the patient and into the cleansing chamber, the blood passes from each cleansing chamber (tube) trapping unwanted disease causing agent (such as cancer cells). In some embodiments, as shown in FIG. 1, a pump is used to move the blood through the cleansing chamber. Ultimately, the cleaned blood is returned to the patient. In some embodiments, the tubes are pre-coated with a coating material. In some embodiments the tubes are coated by flowing various chemicals and biomolecules including binding agents through the tubes before connecting the device to the patient. In some embodiments the tubes include barriers (constriction areas) (603) to make cells and flowing material collide with the tube walls or barriers in order to increase the probability of capture. According to an embodiment of the present invention, the tubes are flexible. In a preferred embodiment, the tubes are spiral or otherwise meandering in shape. In alternate embodiments, the tubes may be rigid and straight in shape. One of ordinary skill in the art would appreciate there any many suitable designs for a tube, and embodiments of the present invention are contemplated for use with any such tube design.

According to an embodiment of the present invention, after treatment is completed, the cleansing chamber (for example the tube or tubes) can be used to analyze the remaining cells via florescent tagging or imaging or other techniques such as cytometry. Similarly ELISA, fluorogenic, electrochemiluminescent, or chromogenic reporters or substrates that generate visible color change to pinpoint the existence of antigen or analyte may be used to analyze the sample. In some embodiments, heat treatment of blood may also be performed. For example, applying heat of a specific temperature may be useful to destroy unwanted cells or other material. In some embodiments, medications, drugs, chemicals or any combination thereof may be added to attack the disease causing agent. In some embodiments, the drugs are removed before the blood is returned to the body. In a preferred embodiment, the drug removal is done by filtering or other methods like the ones described in this disclosure. In some embodiments, radiation may also be used in the cleansing process (903). In some embodiments, radiation (903) is one or more radiations selected from a group of types of radiations comprising waves, particles, electro-magnetic radiation, gamma radiation, radio waves, visible light, and x-rays, particle radiation, alpha particle, beta particle, neutron radiation, acoustic radiation, ultrasound, sound.

Various types of cancer including leukemia are addressed this way and the clean blood is reinserted in the patient. In some embodiments, (arrangement shown at the bottom of FIG. 6) multiple micro-tubes are used. As previously these micro-tubes are functionalized with coating material (such as capturing, binding, or killing) (602). The small size of the cleansing chamber increases the capturing possibility, while the large number of the small size tubes in parallel does not hinder the throughput. In the preferred embodiment, suitable diameters for a tube include, but are not limited to, 10 micron, 20 micron, 30 micron, 50 micron, 100 micron, 500 micron, 1 mm, or less than 2 mm.

A cleansing chamber is one or more cleansing chambers selected from a group of cleansing chambers comprising tube, cylindrical shape, parallelepiped with hollow interior, rectangular parallelepiped. In some embodiments, the parallelepiped design includes a hollow interior with a height of 0.5 mm and a width and length 1 meter by 1 meter, with an inlet and an outlet. In other embodiments the height is 1 mm. In some embodiments the design includes a plurality (multiple) channels running in parallel or meandering, but joining at the inlet and the outlet. In the preferred embodiment, the height on the channels is 0.5 mm or 1 mm, the length of the channels is 1 mm, and the width is 1 mm. In another embodiment, the cleansing chamber is of cylindrical shape packed with spheres. In some embodiments, said spheres are 100 micron in diameter and are coated with coating material. In some embodiments the cleansing chamber is transparent.

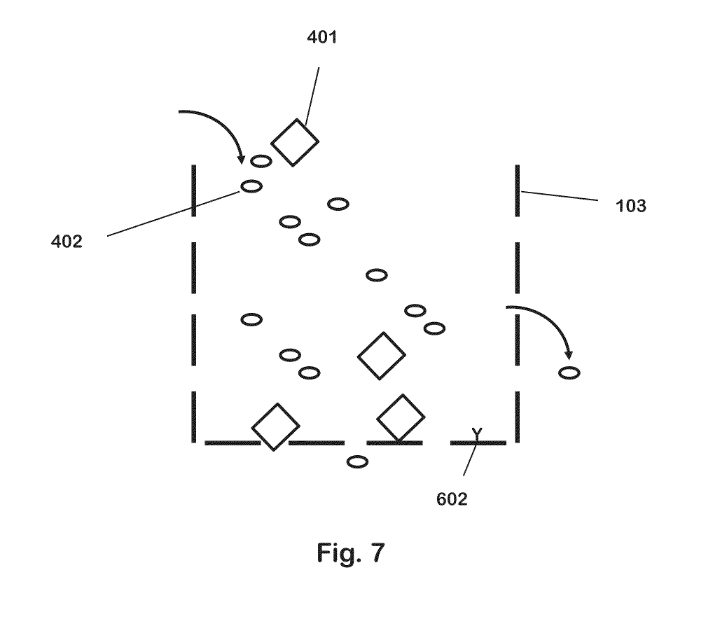

According to an embodiment of the present invention, as shown in FIG. 7, a cleansing chamber that uses filtering is used to separate wanted (402) from unwanted material in the blood. As in illustrative example, CTCs are larger than blood cells. In some embodiments, a coating material (for example binding biomolecule) (602) such as an antibody is coated on the walls of the cleansing or on the filter so that the unwanted (401) particle is captured. In some embodiments osmosis is used (much like in dialysis). In some embodiments the filter is made of microfabricated material, including, but not limited to PDMS or other material like polyimide with micron size holes (e.g. example 10 micron size holes). In some embodiments the blood is cleaned and then returned to the patient (i.e. removal of blood, cleaning, and reinjection). In another embodiment blood is transfused to the patient. Alternatively, blood is mixed with functionalized microbeads with conjugated antibodies or coating material. In some embodiments several beads with different coating material such as antibodies are included. In the preferred embodiment, the cells or material that are to be removed bind to the functionalized beads. As the cells flow, the cells are trapped by the filter because the cells are larger than the opening in the filter. In some embodiments, blood is mixed with the beads in a separate container and then the mixture is inserted in the cleansing chamber.

As an illustrative example, CTCs are larger than other cells in the blood such as leukocytes, red blood cells, and platelets. For instance, CTCs may have diameters 12-25 microns, therefore a 10 micron opening in the filter may block CTCs from going through, while allowing blood cells, which are 90% smaller, to pass through. In some embodiments centrifugation is used to separate cells with the centrifugal force based on density. Alternatively, hydrodynamic sorting is used. One of ordinary skill in the art would appreciate that many filtering methods exist to enhance the removal of unwanted material from the blood, and embodiments of the present invention are contemplated for use with any such filtering method or any combination thereof.

CTCs are captured using specific antibodies able to recognize specific tumor markers such as EpCAM. In some embodiments of the present invention the spheres, tubes, pillar, filters, or walls (or any combination thereof) of the cleansing chamber are coated with a polymer layer carrying biotin analogues and conjugated with antibodies anti EpCAM for capturing CTCs. After capture and completion, therapy images can be taken to further diagnose disease progression by staining with specific fluorescent antibody conjugates. Antibodies for CTC capture include, but are not limited to, EpCAM, Her2, PSA.

According to an embodiment of the present invention, as shown in FIG. 6, the cleansing chamber is composed of tubes (103), for example flexible tubes, coated with coating material (603) such as adhesion protein. The tube is made of a material selected from the group of materials consisting of, but not limited to, glass, quartz, plastic, PDMS, SU-8, polyimide, paralyne, metals, iron, iron oxides, or other materials. In some embodiments the tube is transparent. In some embodiments, the inner surface of the cleansing chamber (example tube) is modified to be receptive to the coating material, for example to a specific antibody or peptide coating. In some embodiments, the cleansing chamber (such as a simple tube) is coated with peptides. In some embodiments, the patient's blood flows through the cleansing chamber (such as a simple tube), but then flow is stopped so that the relevant disease causing agent is allowed to adhere to the coating material on the surface of the cleansing chamber. Next, the blood is flown out of the cleansing chamber (such as a simple tube) after given enough time to maximize capturing. In some embodiments, the blood may be flowed back out of the cleansing chamber after thirty (30) to sixty (60) minutes. In alternate embodiments, the blood may be flowed back out the cleansing chamber after a longer or shorter period depending upon the amount of time required to collect the unwanted material. In some embodiments, the flow rate is 0.5 mL/min. One of ordinary skill in the art would appreciate this amount could be adjusted accordingly based on the particular application. In some embodiments the tube has a spiral shape, while in others the tube has a stacked spiral shape. One of ordinary skill in the art would appreciate that there are many suitable shapes for a tube, and embodiments of the present invention are contemplated for use with any such tube shape.

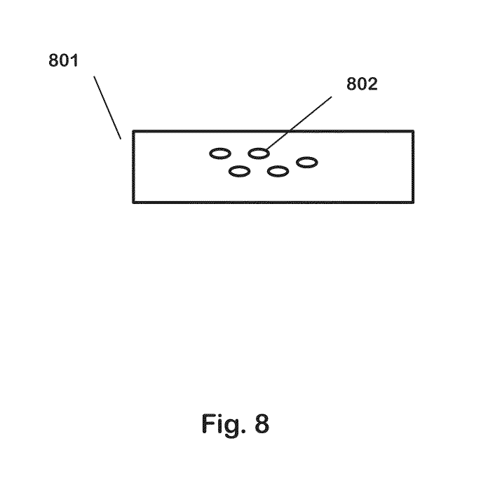

According to an embodiment of the present invention, as shown in FIG. 8, a cleansing chamber 801 with captured material 802 (such as cancer cells) are previously fluorescently tagged with florescent die. For example, FITC labeled antibody is used to tag the cells that have been captured in the cleansing chamber. Next, the florescent cells are counted. In some embodiments an automated system is used to count the cells. The system may include a software system and CCD camera to count the cells. In some embodiments, the entire cleansing chamber is counted. For example, the florescent cells attached to the inner part of the tube are counted by examining the tube outer part. The tube may be rotated to enumerate the cells on all the sides of the tube. In some embodiments, an area is counted and the total number of cells captured is extrapolated from the cell count. In some embodiments the counting is conducted after the capture is completed and the rest of the fluids such as whole blood are removed. One of ordinary skill in the art would appreciate that there are numerous methods to tag and count the cells that are captured, and embodiments of the present invention are contemplated for use with any such method.

According to a first preferred embodiment of the present invention, there is continuous flow through the cleansing chamber. In an alternate preferred embodiment, the cleansing chamber is filled with blood and the flow is stopped for a specific time (for example for 30 minutes), then flow is resumed until the cleansing chamber is full again and the step is repeated.

According to an embodiment of the present invention, the cleansing chamber is exposed to radiation for radiation therapy in order to kill the disease causing agent (e.g. cancer cells or other materials and cells that are malignant). In some embodiments, chemotherapy agents are coated on the surface of the cleansing chamber. As cells flow through the cleansing chamber they collide with the surface of the cleansing chamber and die or attach and die if antibody capturing is also used in combination with chemotherapy agents. In some embodiments chemical substances, such as one or more anti-cancer drugs, are used. In some embodiments, drugs that are not indiscriminately cytotoxic (such as monoclonal antibodies) are coated on the surface of the cleansing chamber. These drugs target specific proteins expressed specifically on the cells that have to be removed, such as proteins on a bacterium or cancer cell.

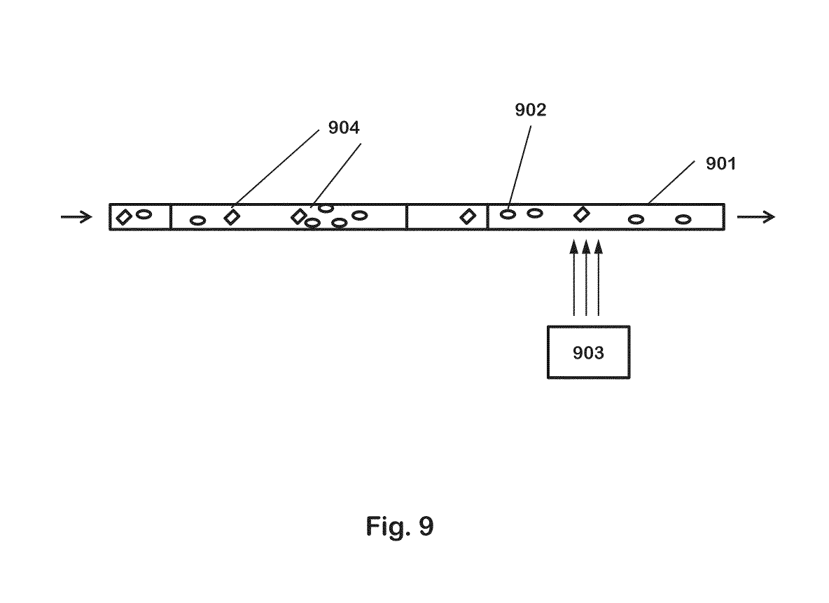

According to an embodiment of the present invention, as shown in FIG. 9, light exposure 903 is included in a way such that the cleansing chamber 901 is exposed to light to achieve photochemotherapy (also referred to as photodynamic therapy or PDT). In a preferred embodiment, the disease causing agent 904 is destroyed by administering a photosensitizer material intravenously. A photosensitizer is a light-sensitive compound that becomes toxic and generates reactive oxygen species (ROS) from oxygen molecules when exposed to light of a specific wavelength. Different photosensitizers have different activation wavelengths at which they become reactive. In the preferred embodiment, the photosensitizer is linked to a binding agent such as an antibody or peptide that attaches selectively to the disease causing agent and the disease causing agent flows along with the blood through the cleansing chamber. Light is then delivered to the disease causing agent as it passes through the cleansing chamber to cause the destruction of the disease causing agent. Photosensitizers are functionalized to specifically attach to the above mentioned targets. Examples of photosensitizers include, but are not limited to: ce6, chlorophylls, porphyrins, dyes, Silicon Phthalocyanine Pc 4, aminolevulinic acid, mono-L-aspartyl chlorine, m-tetrahydroxyphenylchlorin (mTHPC). In some embodiments the photosensitizer is linked to a binding material, such as antibody or peptide, that is attached to the inner walls of the cleansing chamber (such as the inner tube). The disease causing agent 904 flows along with blood 902 through the cleansing chamber 901. Then, the disease causing agent attaches to the binding material (antibody or peptide) linked to the photosensitizer. Light is then delivered to cause the destruction of the disease causing agent.

According to an embodiment of the present invention, hyperthermia therapy may be used to aid in the cleansing of the blood. In a preferred embodiment, once blood is flown through the cleansing chamber it is heated to high enough temperatures so as to cause apoptosis or cell death or otherwise destroy or deactivate the target. In the preferred embodiment, heating can be conducted in active flow or without blood flow (e.g. the cleansing chamber is filled with blood, the flow is stopped, and then the cleansing chamber is heated). In some embodiments the cleansing chamber is the cooled to normal body temperatures. In some embodiments there are several chambers (compartments) for cooling and heating.

According to an embodiment of the present invention, the cleansing chamber may be used to remove CTCs from the bloodstream aiming at reducing the chances of metastasis. In a preferred embodiment, the cleansing chamber may be a modified commercially available plastic tube that is coated with a binding material such as EpCAM antibodies. In some embodiments, blood flows through a tube where CTCs bind to EpCAM antibodies coated on the inner surface of the tube. In the preferred embodiment, this procedure can be done safely and successfully in a clinical setting by (i) processing the entire blood in continuous circulation or (ii) consecutive drawing of as much as 0.5 liter of blood (a quantity in line with typical blood donations), undergoing the cleaning process for CTC removal, and re-injecting the blood in the patient, then repeating the process until all of the blood is cleaned from CTCs (a typical adult has a blood volume between 4.7 and 5 liters).

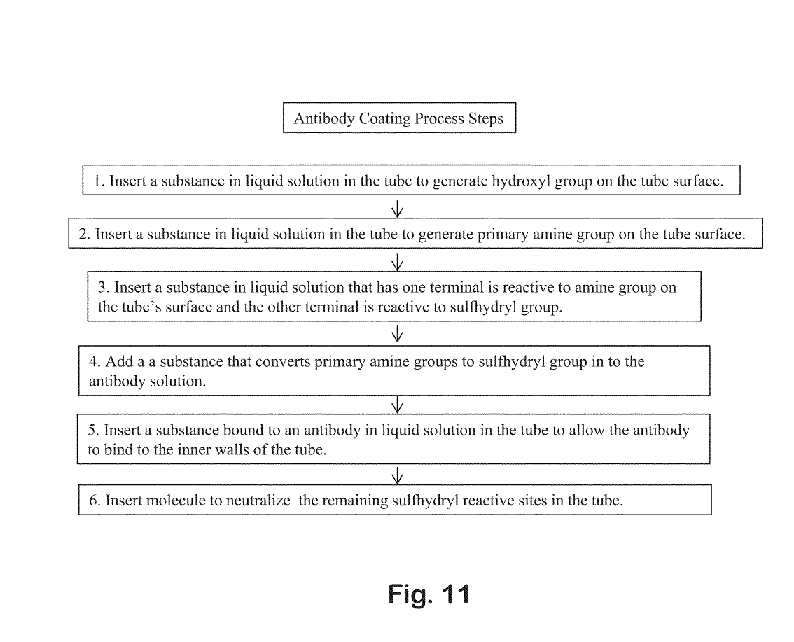

Turning now to FIG. 11, an exemplary process of applying the coating material to the cleansing chamber (including, but not limited to, a tube) comprises the following steps: (1) PDMS tube is treated by hydrogenperoxide (H2O2):hydrochloric acid (HCL):water (H2O) mixture. This treatment can generate hydroxyl group (—OH) on the PDMS tube inner surface. (2) The tube is treated by aminopropyltrimethoxysilane (TMOS) (or aminopropyltriethoxyxilane (TEOS)). This step can produce primary amine group on the tube surface. (3) The tube is filled with Sulfosuccinimidyl-4-(N-maleimidomethyl)cyclohexane-1-carboxylate (Sulfo-SMCC) solution (in buffer at pH 7.4). Sulfo-SMCC is a hetero-bifunctional-crosslinker (one terminal is reactive to amine group and the other terminal is reactive to sulfhydryl group). (4) At the same time, 2-iminothiolane (2-IT) is added to antibody solution and the mixture is stirred at room temperature in a vial (not inside the tube yet). 2-IT converts primary amine groups in the given antibody to sulfhydryl group (—SH). Then, the excess 2-IT is removed from antibody solution by centrifugal filtration and the excess Sulfo-SMCC is removed from the tube (excess Sulfo-SMCC is defined as the Sulfo-SMCC that is not bound to the tube). (5) Product from step 3b, which is the antibody solution, is injected in the tube following step 3a (in step 3a the tube has been treated with Sulfo-SMCC). This step allows the sulfhydryl group on the antibody to react with sulfhydryl reactive terminal of sulfo-SMCC, resulting in antibody coated tube inner surface by covalent linkage. (6) The antibody conjugated tube surface is treated by cystein solution. Cystein (an amino acid with —SH group) can cap the remaining sulfhydryl reactive site of tube and neutralize the electric charge of the tube surface. One of ordinary skill in the art would appreciate that there a number of modifications that could be made to the above described steps without departing from spirit and scope of the present invention.

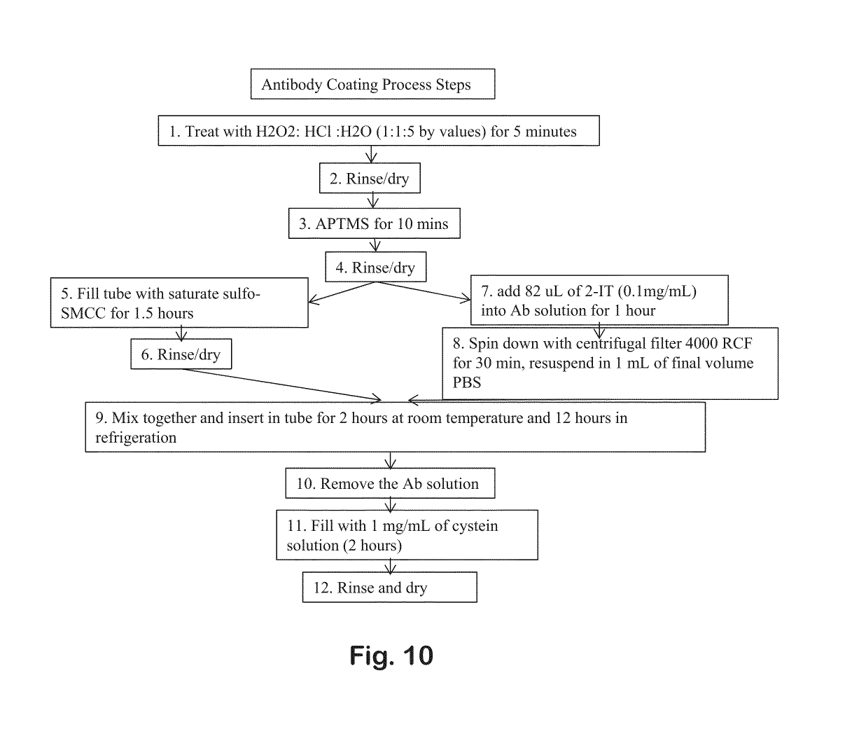

According to an embodiment of the present invention, a polydimethylsiloxane (PDMS) tubing (laboratory tubing with 1.02 mm in inner diameter) can be used. The tube's internal surface is activated by treating with acidic hydrogenperoxide solution (H₂O:HCl:H₂O₂ in 5:1:1 volume ratio) for 5 minutes at room temperature (FIG. 10 step 1). The tube is rinsed with excess deionized (DI) water 5 times and dried in air (FIG. 10 step 2). This treatment forms the hydrophilic surface with hydroxyl groups available for further functionalization. Then, the tube is filled with aminopropyltrimethoxysilane (APTMS) for 10 minutes (FIG. 10 step 3). The tube is rinsed with excess amount of DI water at least 5 times and dried in air. This step adds the primary amine group on the surface based on the sol-gel reaction principle (FIG. 10 step 4). Then, the tube is rinsed and the fluorescence from tube's inner surface is monitored using fluorescence microscope.

EpCAM is a widely accepted CTC marker due to CTC's epithelial origin. Therefore, according to an embodiment of the present invention, EpCAM antibody is treated with Traut's reagent (2-iminothiolane HCl, 2-IT) to generate an available sulfhydryl group (—SH) (anti-EpCAM:2-IT=1:10 in mole ratio) in PBS (pH 7.4) for 1 hour (FIG. 10 step 7). Then, unbound 2-IT is removed from the antibodies using centrifugal filter (MWCO 30 kDa, Amicon filter or Corning Spin-X protein concentrator) at 4000 RCF for 30 minutes (FIG. 10 step 8). The concentrated anti-EpCAM is resuspended in PBS, adjusting the volume of 1 mL. During the antibody-2-IT reaction, the amine functionalized tube is filled with a hetero-bifunctional (amine reactive at one terminal and thiol reactive at the other terminal) cross-linker, sulfo-SMCC (sulfosuccinimidyl 4-[N-maleimidomethyl]cyclohexane-1-carboxylate) in 2 mg/mL concentration in PBS (pH 7.4) (FIG. 10 step 5). After the EpCAM is spinned down, the sulfo-SMCC solution is removed from tube, and the tube is rinsed in PBS and re-filled with 1 mL EpCAM solution (FIG. 10 step 6). The reaction is run for 2 hours at room temperature and kept on going overnight at 4° C. on a shaker (FIG. 10 step 9). The next day, after the unbound EpCAM solution is collected (FIG. 10 step 10), the tube is gently rinsed with PBS and then refilled with 1 mg/mL L-cystein for further 2 hours (FIG. 10 step 1011). The tube is rinsed and dried (FIG. 10 step 1012). The conjugation of anti-EpCAM on the tube surface is confirmed by PE's fluorescence on a fluorescence microscope. One of ordinary skill in the art would appreciate that there a number of modifications that could be made to the above described steps without departing from spirit and scope of the present invention.

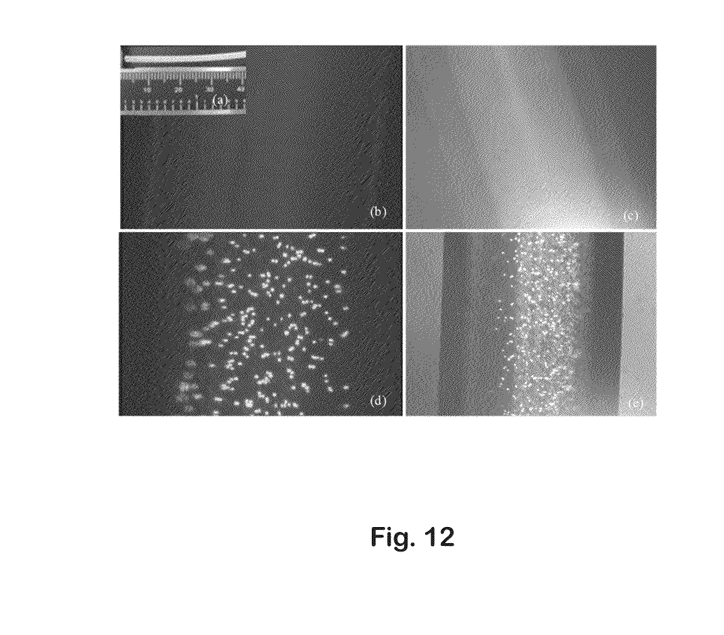

Turning now to FIG. 12, (a) a tube, like the one shown in the picture, are functionalized with human anti-EpCAM (ruler scale in mm) as described above. As shown in (b) and (c), PC-3 cells were placed in an unmodified tube (without EpCAM coating), for control measurements, no capture was observed. As shown in (d) and (e), fluorescent microscopic images of captured PC-3 cells on anti-EpCAM immobilized tube (light areas shown in the tubes). The images in (d) and (e) are of captured PC-3 cells by anti-EpCAM conjugated silicone (PDMS) tube after 1 hour of incubation. After collecting the solution from tube, captured cells were stained with Calcein AM containing cell media and imaged using GFP filter cube (Ex: 485 nm/Em: 525 nm) with an Olympus IMT-2 fluorescence microscope. The result showed that PC-3 cells were effectively captured by the anti-EpCAM immobilized tube. Due to the fact that Calcein AM is a cell viability indicating fluorescent probe, these images also confirm that the captured cells are alive. In contrast the unmodified control tubes, shown in (b) and (c), exhibited negligible capture of PC-3 cells.

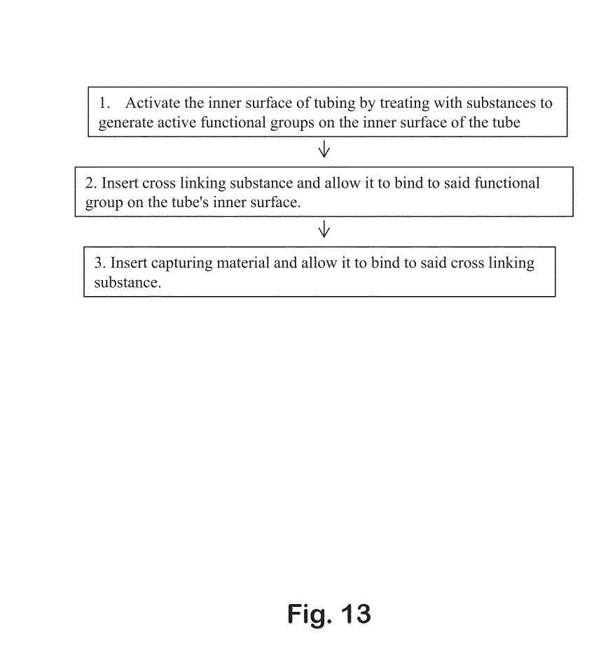

Turning now to FIG. 13, an exemplary process to functionalize cleansing chamber such as a tube for capturing specific substances may comprise the following steps: (1) activate the inner surface of tubing by treating with substances to generate active functional groups on the inner surface of the tube; (2) insert cross linking substance and allow it to bind to said functional group on the tube's inner surface; (3) insert coating material and allow it to bind to said cross linking substance. Said coating material is designed to bind to disease causing agent. According to an embodiment of the present invention substances to generate active functional groups are selected from the group of active functional group generating substances comprising acidic hydrogenperoxide solution (H₂O:HCl:H₂O₂ in 5:1:1 volume ratio), aminopropyltrimethoxysilane (APTMS). According to an embodiment of the present invention cross linking substances are selected from the group of cross linking substance comprising 1-Ethyl-3-[3-dimethylaminopropyl]carbodiimide hydrochloride (EDC or EDAC), sulfo-SMCC (sulfosuccinimidyl 4-[N-maleimidomethyl]cyclohexane-1-carboxylate), polymeric linkers.

According to an embodiment of the present invention, the cleansing chamber is a medical tube. In a preferred embodiment, the tube is selected from a group of tube comprising plastic tubes, polymer tube, metallic tube, silicone tube, glass tubes. In some embodiments, the captured cells on the tube are counted and further re-suspended and genetically analyzed, or re-cultivated. In another embodiment, additional filters and apoptosis causing agents are added to enhance the capture/kill rate. In another embodiment, the system is part a dialysis machine. In another embodiment, a machine that includes the tube also includes anticoagulant inlets, filters to filter cells by size (for example 25 um size separation holes), and photodynamic therapy. In some embodiments a dialysis membrane is added to remove microorganisms by their smaller size.

The elimination of disease causing agent such as circulating tumor cells from the blood stream is achieved by flowing the blood though an extracorporeal tube and applying photodynamic therapy (PDT). In an embodiment of the claimed invention, an extracorporeal PDT (also known as photoimmunotherapy in conjunction with antibody targeting) is used to treat a patient by eliminating blood-borne disease causing agent. Specifically, a photosensizer, is conjugated to an antibody in order to target disease causing agent. As the blood circulates through a transparent medical tube, it is exposed to light of a specific wavelength generated by an LED array such as 660 nm wavelength. In some embodiments, a 2 minute exposure is sufficient to achieve selective cancer cell necrosis. PDT is performed while the blood is in circulation. One of ordinary skill in the art would appreciate that there a number of photosensitizers that could be used in the above described steps without departing from spirit and scope of the present invention, including photosensitizers that have different activation wavelengths for ROS generation. One of ordinary skill in the art would appreciate that a photosensitizer may not require conjugation to an adhesion molecule.

According to an embodiment of the present invention, a method for preparing a cleansing chamber, such as a tube to be used for capturing disease causing agent, comprises the steps of: activating an inner surface of the tube by treating the inner surface with substances to generate active functional groups on the inner surface of the tube; inserting into the tube a crosslinking substance such that the crosslinking substance binds to said functional group on the inner surface of the tube; inserting coating material into the tube such that the coating material binds to said crosslinking substance, wherein said coating material is designed to bind to said substances. In a preferred embodiment, possible tubes that could be used with the method include, but are not limited to plastic tubes, polymer tubes, metallic tubes, and silicone tubes. In the preferred embodiment, the substances that generate active functional groups include, but are not limited to, acidic hydrogenperoxide solution (H₂O:HCl:H₂O₂ in 5:1:1 volume ratio) and aminopropyltrimethoxysilane (APTMS). In the preferred embodiment, possible crosslinking substances include, but are not limited to, 1-Ethyl-3-[3-dimethylaminopropyl]carbodiimide hydrochloride (EDC or EDAC), sulfo-SMCC (sulfosuccinimidyl 4-[N-maleimidomethyl]cyclohexane-1-carboxylate), polymer, polymeric linker and Polyethylene Glycol (PEG). In the preferred embodiment, possible coating materials include, but are not limited to antibodies, aptamers, peptides, polymers, proteins, nucleic acid, RNA, DNA, organic materials and magnetic particles.

According to an embodiment of the present invention, a method for preparing a cleansing chamber, such as a tube to be used for capturing disease causing agent, comprises the steps of: activating an internal surface of the tube by treating the internal surface with an acidic hydrogenperoxide solution to form a hydrophilic surface with hydroxyl groups; filling the tube with aminopropyltrimethoxysilane to add a primary amine group on the internal surface; treating an antibody with a solution to generate available sulfhydryl group (—SH); filling the tube with a hetero-bifunctional cross-linker; removing the excess hetero-bifunctional cross-linker solution from tube; filling the tube with the antibody solution; and filling the tube with L-cystein.

PDT functions to destroy (or at least damage) cells or tissues by employing a photosensitizer. Such photosensitizer interacts with light (primarily in the visible range) to generate reactive oxygen species (principally singlet oxygen, ¹O₂). Toxicity of the reactive oxygen species is localized to the cell in direct contact with it, due to the singlet oxygen's short (<100 nm) diffusion distance. This characteristic results in high specificity to the targeted (diseased) cell with near zero collateral damage to adjacent cells/tissues, making PDT an effective and safer treatment compared to conventional radiation and chemotherapy. In spite of these advantages, PDT is limited to applications in opened/topical regions including skin, head, neck, lungs, and teeth because visible light can barely penetrate through tissue, especially in the presence of blood (a visible light absorber) and water (an IR light absorber) However, in this invention PDT is performed in a transparent cleansing chamber such as a tube, thereby providing the necessary light to generate damage-causing reactive oxygen species. In an exemplary embodiment, PDT is performed by flowing blood through a cleansing chamber such as a thin transparent medical tube, the transparency and thinness of which allows light to penetrate through the chamber for activating the photosensitizer.

In an embodiment of the claimed invention, a photosensitizer-antibody conjugate is used to selectively deliver the photosensitizing agent to disease causing agents. A benefit to this technique is that the antibody can be safely cleared out of the body by natural antibody degradation mechanisms within a few days.

According to an embodiment of the claimed invention, the photosensitizer Chlorin E6 (Ce6) is conjugated to the antibody CD44 (human). Ce6 is a naturally occurring, commercially available photosensitizer that has excitation maxima in the far-red/near IR region (around 667 nm) and relatively high quantum efficiency. Because the Ce6 molecule has three carboxyl groups, it can be readily modified for chemical conjugation. To conjugate the photosensitizer to the antibody, 2 mg of Ce6 is mixed with 6.5 mg of crosslinker, 1-Ethyl-3-[3-dimethylaminopropyl]carbodiimide hydrochloride (EDC) and 7.6 mg of sulfo-NHS in 1 mL PBS buffer at pH 7.4 (at 1:10:10 mole ratio respectively). The reaction is incubated for 2 hour at room temperature. Then, 50 μL, of the solution is added to 100 μL, of FITC labeled human CD44 antibody solution. The solution is further incubated for 3 hours at room temperature with agitation. The reaction mixture is spin-filtered to remove the unbound Ce6 at 4000 RCF for 100 min. The final Ce6-CD44 Ab product is resuspended in PBS, adjusting the total volume of 100 μL and stored at 4° C.

PDT is an effective alternative treatment modality, which addresses several of the drawbacks of conventional treatments in cancer and in other diseases. However, the absorption of visible light by blood (especially due to the red blood cells' hemoglobins) significantly reduces the penetration of light through tissue. In this invention the use of a transparent tube improves the outcomes. In some embodiments the tube used is a transparent PDMS tube with 1 mm inner diameter. Since the light comes from all the directions surrounding the tube in a reflective chamber, the thin diameter of tube allows for nearly the entire sample to be within the penetration depth of light. More exposure to light results in better outcomes with PDT.

In another embodiment, the photosensitizer-antibody conjugates are used as an imaging agent to detect metastasized cancers, allowing other treatment modalities, including endoscopic photodynamic therapy. In another embodiment, the lymphatic system is targeted.

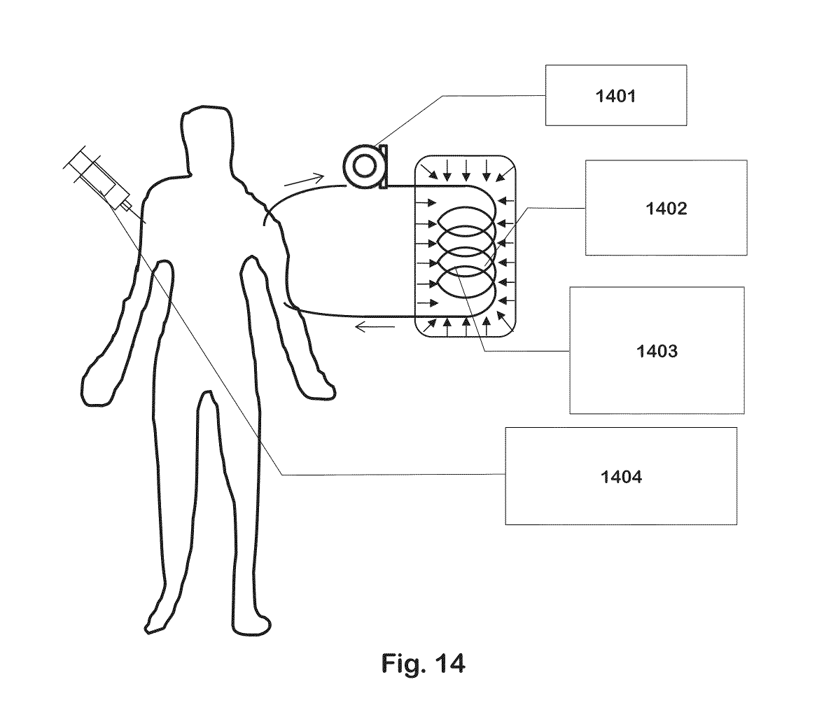

FIG. 14 is a schematic of the proposed cleansing chamber in operation. A photosensitizer or photosensitizer-antibody conjugate (1404) is injected prior to PDT procedure and certain time is allowed for the conjugate to bind with the disease causing agent. Blood circulation was guided by medical tubing with a peristaltic pump (1401). Extracorporeal PDT is performed as the blood flows through the tube inside an illumination chamber (providing light at 660 nm wavelength). The treated blood is returned to body. In some embodiments all procedures are in constant flow. The extracorporeal circulation path (the tubing) (1403) is shown.

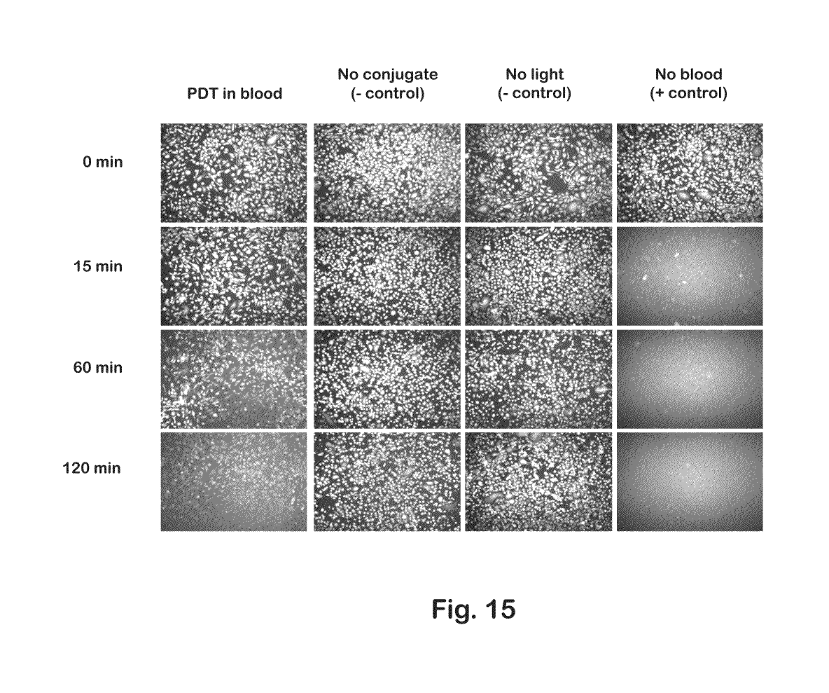

FIG. 15 shows results of the efficacy of photodynamic therapy after 2 minutes illumination on a plate in the presence and absence of blood. Cancer cells are stained with Calcein AM. The figure demonstrates that PDT is not effective in the presence of light absorbing light. The rightmost column reveals significant cell death population when target cells are exposed to light, demonstrating the efficacy of PDT therapy in general. However, the leftmost column reveals how PDT effectiveness is significantly hampered when light-absorbing blood is present.

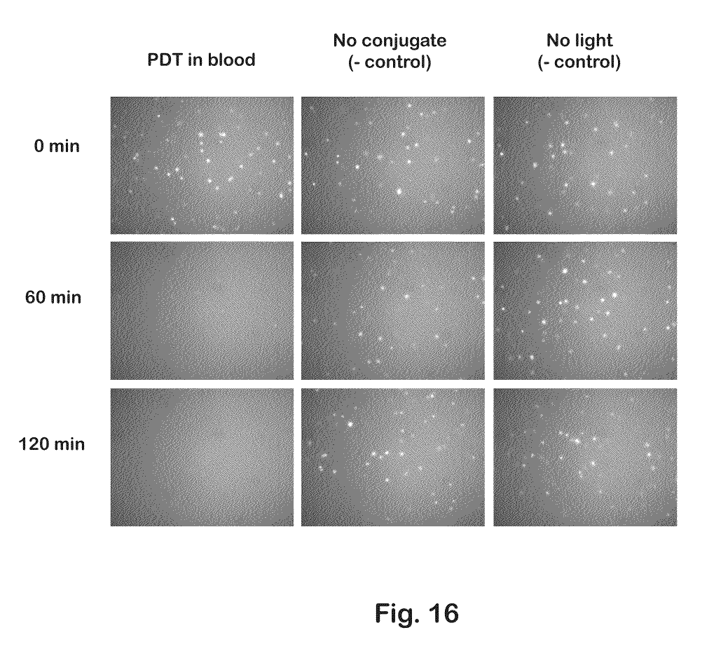

FIG. 16 shows how photodynamic therapy is effective in a tube with 2 min illumination. The tube's inner diameter is 1.02 mm, which is within the penetration depth of light given that the tube is illuminated from all directions. Since targeted cells are exposed to light, there is significantly more cell death compared to the results in light-absorbing media mimicking blood, as shown in the left hand side “PDT in blood” column.

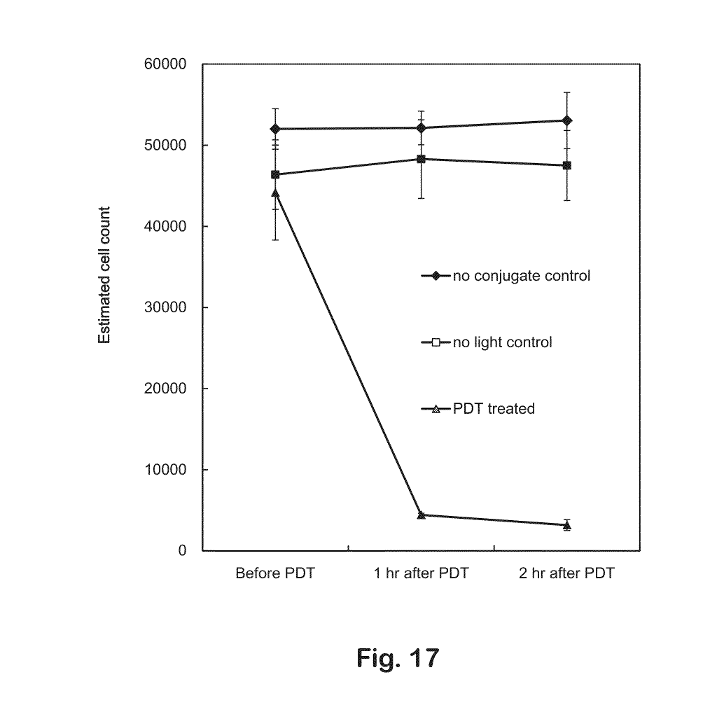

FIG. 17 shows results the quantitative analysis of PDT outcome for PC-3 cells in tube. (n=3, data represent mean±standard error).

In some embodiments a photosensitizer is conjugated to binding agent (also called binding material, in this disclosure “antibody” is used, however the term can be replaced with any other binding agent), such as an antibody, protein, peptide, molecule, or material that binds to the pathogen or the cell that is being targeted. In some embodiments a crosslinker is used to modify the photosensitizer and make it receptive to the binding agent. Then this is mixed with the binding agent. In some embodiments the conjugation reaction is run for several hours at room temperature with agitation. In some embodiments the reaction mixture is spin-filtered to remove the unbound photosensitizer. The photosensitizer-binding agent conjugate is injected in the patient. A method is used to access the blood by: an intravenous catheter, or an arteriovenous fistula (AV) or a synthetic graft. In some embodiments a pump is used. Blood circulates through medical tubing, partially resting inside a chamber. In some embodiments the chamber is illuminated. Light of a specific wavelength illuminates inside the chamber and activates the photosensitizer. In some embodiments the tube is modified with additional binding agent to capture the pathogen or the cell. In some embodiments a filter is also used to filter by size. In another embodiment sonodynamic therapy or other forms of therapy are used in addition to the therapy disclosed herein.

In this disclosure a photosensitizer is a compound that is excited when it absorbs light of a specific wavelength. The excitation creates a energy transfer to oxygen to produce singlet oxygen. Singlet oxygen attacks any organic compounds nearby and is able to destroy cells. According to an embodiment of the present invention, wherein the photosensitizer is selected from the group of photosensitizers: aminolevulinic acid (ALA), Silicon Phthalocyanine Pc 4, m-tetrahydroxyphenylchlorin (mTHPC), and mono-L-aspartyl chlorin e6 (NPe6), Allumera, Photofrin, Visudyne, Levulan, Foscan, Metvix, Hexvix, Cysview, and Laserphyrin, Antrin, Photochlor, Photosens, Photrex, Lumacan, Cevira, Visonac, BF-200 ALA, Amphinex, Azadipyrromethenes, Methylene Blue. Photosensitizers are all fluorescent dyes. They are largely classified in porphyrin based photosensitizers and non-porphyrin based photosensitizers. One of ordinary skill in the art would appreciate that there are other photosensitizers can be used without departing from spirit and scope of the present invention.

According to an embodiment of the present invention, a method for removing disease causing agent from blood that comprises the steps of: attaching a photosensitizer to a binding agent to generate a conjugate material; injecting the conjugate material into a patient such that the conjugate material binds to a disease causing agent; circulating blood through an extracorporeal transparent tube; illuminating said tube with light to activate said photosensitizer, wherein the activation of said photosensitizer generate reactive oxygen capable of causing cell death upon contact. In the preferred embodiment, the extracorporeal transparent tube is selected may be any tube suitable for the application including, but not limited to, plastic tubes, polymer tubes, metallic tubes, silicone tubes. In the preferred embodiment, the extracorporeal transparent tube has an inner diameter of 1 mm. In the preferred embodiment, the extracorporeal transparent tube is modified with one or more additional binding agents to capture said disease causing agent. Possible binding agents for use in the preferred embodiment include, but are not limited to, an antibody, a protein, a peptide, a molecule, or one or more of a material that binds to a pathogen, a cell, or a cancer cell. In the preferred embodiment, the photosensitizer is modified with a crosslinker to make it receptive to a binding agent. In the preferred embodiment, the wavelength of light to activate the photosensitizer is 660 nm. In the preferred embodiment, the conjugate material is used as an imaging agent.

In some embodiments, these procedures and therapies and systems are used during a surgical procedure to remove disease causing agent. In other embodiments these therapies and processes and systems are used as part of an ongoing therapy regime. As an illustrative example, a patient could undergo a cleansing procedure multiple time per week using the methods deribed herein. These methods may be combined with other therapies such as sonodynamic therapy, where ultrasound activated therapy similar to PDT is also used to attack a disease causing agents.

In some embodiments, these techniques are used in conjunction with other therapies to increase the chances of survival and minimize the changes of metastasis. The present invention may be used during primary tumor removal surgery, post or pre surgery, or in lieu of surgery. The present invention may also be used during hemodialysis or following hemodialysis. In some embodiments the method and device may be used inside of a hemodialysis unit. In some embodiments the device may be configured to specifically target MRSA.

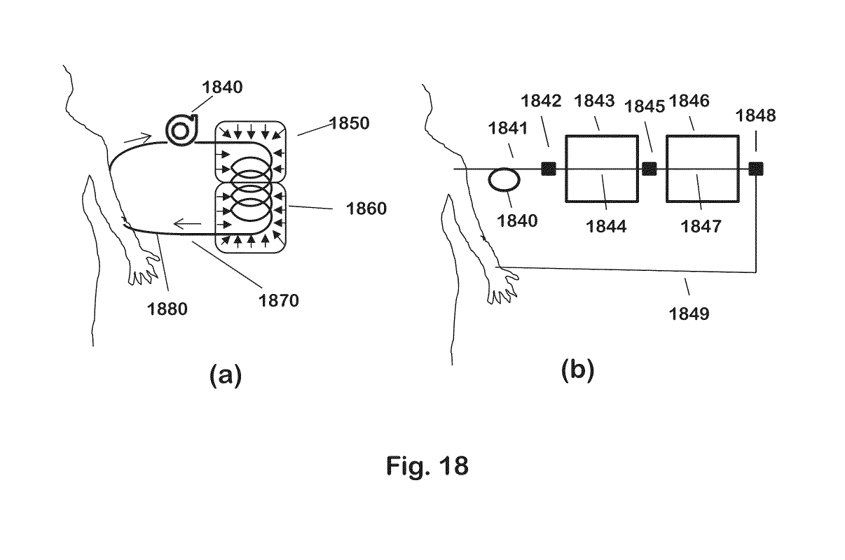

According to an embodiment of the present invention, a method and apparatus for blood borne pathogen removal that involves capturing and killing pathogens by flowing the blood of an infected individual through a cleansing chamber and circulating the cleansed blood back to the individual. Three independent techniques and their combinations are disclosed and shown together in FIG. 18. The techniques include (a) a cleansing chamber such as a chemically modified medical tube for capturing and removing pathogens, (b) a photosensitizer that adheres to the pathogens while in circulation (in some embodiments by conjugating the photosensitizer with an antibody) and is activated by near-IR light when the blood flows through a cleansing chamber such as an extracorporeal tube, the photosensitizer kills the pathogens by releasing ROS, and (c) a cleansing chamber, such as an extracorporeal tube, that is exposed to a light source with UV-light to kill pathogens.

Turning now to FIG. 18 (a), a first embodiment of a conceptual diagram of an extracorporeal cleansing chamber is shown. In the preferred embodiment, the blood is circulated through a cleansing chamber, such a tube, using a pump, for instance a peristaltic pumping, while a medical tube circulates the blood of a patient. A patient is injected with photosensitizer. A pump (1840) helps circulate the blood into a chamber where a light source exposes the cleansing chamber to near-IR (wavelength˜660 nm) (1850) and UV (wavelength 400 nm-100 nm) (1860) light. Next, the blood moves through a second cleansing chamber with coating material, for instance a functionalized tube (1870) for capturing the targeted pathogen or pathogens. A photosensitizer-antibody conjugate may be administered through the administration port (1880).

Turning now to FIG. 18 (b), a second embodiment of a conceptual diagram of an extracorporeal cleansing chamber is shown. In the preferred embodiment, the cleansing chamber may be cooled or placed inside another chamber with lower temperature, for instance at a temperature of 4 Celsius. In some embodiments, only the section of the cleansing chamber that is coated with coating material is cooled. In the preferred embodiment, the blood goes through the first tube (1841). A pump (1840) is used to circulate the blood through the first cleansing chamber (1844) coated with coating material. The first tube (1841) is connected to the first cleansing chamber (1844) via a tube connector (1842). The cleansing chamber (1844) resides partially or entirely inside a cooling chamber (1843). Another connector (1845) connects the first cleansing chamber (1844) to a second cleansing chamber (1846) where a light source (1846) exposes it to light of a specific wavelength defined elsewhere in this disclosure. Then via another connector (1848) to a tube (1849) the blood is returned cleaned.

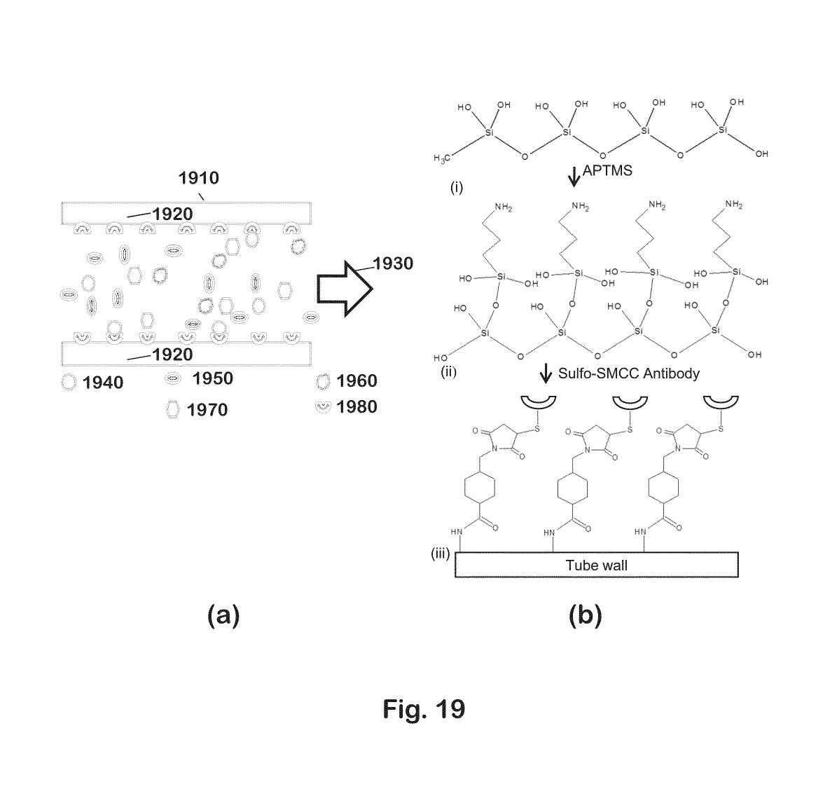

Turning now to FIG. 19, a conceptual diagram of capturing pathogens with coating material, such as antibody immobilized on cleansing chamber wall, is shown. A cleansing chamber with coating material (such as a functionalized tube) (1910) is shown. The tube wall (1920) is this example is coated with coating material which is an adhesion molecule (such as an antibody) or pathogen killing molecule (1980). In the preferred embodiment, as blood flows (1930) the pathogens (1940) are captured or killed by the pathogen killing molecules (1980), while the red blood cells (1950), platelets (1960), white blood cells (1970) flow back to the patient.

According to an embodiment of the present invention, the cleansing chamber is a polydimethylsiloxane (PDMS) tubing. In a preferred embodiment, the tubing has an internal diameter of 1.02 mm, but may have a wider or narrower diameter based on a given application. In the preferred embodiment, the cleansing chamber is prepared as follows: the cleansing chamber's internal surface is activated by treatment with an acidic hydrogen peroxide solution (H2O:HCl:H2O2 in 5:1:1 volume ratio) for five minutes at room temperature. Then, the cleansing chamber is rinsed with excess deionized (DI) water five times and dried in air. This treatment leads to the hydrophilic surface with hydroxyl groups (—OH) available for further functionalization. The cleansing chamber is then filled with aminopropyltrimethoxysilane (APTMS) for 10 minutes. Next, the cleansing chamber is rinsed with excess DI water at least five times and dried in air. This final step adds the primary amine group on the surface based on the sol-gel reaction principle. To verify the presence of the primary amine group on the tube surface, a short section of the treated cleansing chamber is filled with an amine reactive fluorescence dye, fluorescein isothiocyanate (FITC, 0.1 mg/mL in PBS pH 7.4) for one hour. The cleansing chamber is then rinsed, and the fluorescence from its inner surface is monitored using a fluorescence microscope. An antibody specific to the microorganism that is targeted is treated with a reagent such as (2-iminothiolane HCl, 2-IT) to generate an available sulfhydryl group (—SH) (antibody:2-IT=1:10 in mole ratio) in PBS (pH 7.4). Then, unbound reagent (such as 2-IT) is removed from the antibodies using a protein concentrator (MW cut off 30 kDa, Corning Spin-X protein concentrator) at 5000 RCF for 30 minutes. The concentrated antibody is re-suspended in PBS, and the volume is adjusted to fill the cleansing chamber. During the antibody-reagent reaction, the amine functionalized tube is filled with a hetero-bifunctional crosslinker, sulfo-SMCC (sulfosuccinimidyl 4-[N-maleimidomethyl]cyclohexane-1-carboxylate) in 2 mg/mL concentration in PBS (pH 7.4). Following a spinning down, the sulfo-SMCC solution is removed, and the cleansing chamber is rinsed in PBS and re-filled with resuspended antibody solution. The reaction is run on a shaker for two hours at room temperature and continued overnight at 4° C. The next day, after the unbound antibody solution is collected, the cleansing chamber is gently rinsed with PBS and then refilled with 2 mg/mL L-cysteine for another two hours. The conjugation of antibody on the tube surface is confirmed by FITC labeling on a fluorescence microscope. In this example antibody was used for a coating material and a tube for a cleansing chamber. Other coating materials and types of cleansing chambers can also be used. One of ordinary skill in the art would appreciate that the steps of this process could be modified depending on a given application or procedure, in each case without departing from the spirit and scope of the method described.

Turning now to FIG. 19(b) a polydimethylsiloxane (PDMS) tubing (e.g. Dow Corning Silastic laboratory tubing with an internal diameter of 1.02 mm) may be used in accordance with an embodiment of the method described herein. As an illustrative example the tube length may be around about 120 cm. In the preferred embodiment, the internal surface of the tube is activated by treatment with an acidic hydrogen peroxide solution (H2O:HCl:H2O2 in 5:1:1 volume ratio) for five minutes at room temperature. Then, the tube is rinsed with excess deionized (DI) water five times and dried in air. This treatment forms the hydrophilic surface with hydroxyl groups (—OH) available for further functionalization (FIG. 19 (b) (i)). The tube is then filled with aminopropyltrimethoxysilane (APTMS) for 10 minutes (FIG. 19 (b) (ii))). Next, the tube is rinsed with excess amount of DI water at least five times and dried in air. This step adds the primary amine group on the surface based on the sol-gel reaction principle. To verify the presence of the primary amine group on the tube surface, a short section of the treated tube is filled with an amine reactive fluorescence dye, fluorescein isothiocyanate (FITC, 0.1 mg/mL in PBS pH 7.4) for one hour (FIG. 19 (b) (ii)). Then, the tube is rinsed and the fluorescence from its inner surface is monitored using a fluorescence microscope. Immobilization of antibody like anti-EpCAM on the surface of the tube is done as follows: in this example Phycoerythrin (PE)-labeled human EpCAM (eBiosciences) antibody (however this process is used with other coating materials as well) is treated for one hour with Traut's reagent (2-iminothiolane HCl, 2-IT) to generate an available sulfhydryl group (—SH) (anti-EpCAM:2-IT=1:10 in mole ratio) in PBS (pH 7.4). Next, unbound 2-IT is removed from the antibodies using a spin column (MW 30 kDa, cutoff, Amicon filter or Corning Spin-X protein concentrator) at 4000 RCF for 30 minutes. Then the concentrated anti-EpCAM is re-suspended in PBS, and the volume adjusted to 1 mL. During the antibody-2-IT reaction, the amine functionalized tube is filled with a hetero-bifunctional (amine reactive at one terminal and thiol reactive at the other terminal) cross-linker, sulfo-SMCC (sulfosuccinimidyl 4-[N-maleimidomethyl]cyclohexane-1-carboxylate) in 2 mg/mL concentration in PBS (pH 7.4). After the EpCAM is spun down, the sulfo-SMCC solution is removed and the tube is rinsed in PBS and re-filled with 1 mL EpCAM solution. The reaction is run on a shaker for two hours at room temperature and continued overnight at 4° C. The next day, after the unbound EpCAM solution is collected, the tube is gently rinsed with PBS and then refilled with 1 mg/mL L-cystein for another two hours (FIG. 19 (b) (iii)). The conjugation of anti-EpCAM on the tube surface is confirmed by PE's fluorescence on a fluorescence microscope.

In some embodiments of photodynamic therapy, a photosensitizer such as Chlorin E6 (Ce6) is used. In this embodiment, because the Ce6 molecule has three carboxyl groups, the photosensitizer will need to be modified. To modify, the Ce6 is mixed 1-Ethyl-3-[3-dimethylaminopropyl]carbodiimide hydrochloride (EDC, crosslinker) and sulfo-NHS (stabilizer for EDC) in 10% Dimethyl sulfoxide-PBS buffer (DMSO:PBS=10:90), (Ce6:EDC:sulfo-NHS=1:10:10 in mole ratio). The reaction is run at room temperature with agitation for 2 hours. Then, an antibody in 10% DMSO-PBS mixture is mixed with 1 mL of Ce6 mixture. The conjugation reaction is run at room temperature with agitation for 3 hours. The reaction mixture is spin-filtered with a protein concentrator to remove the unbound Ce6 and other chemicals from the desired Ce6-antibody conjugates at 5000 RCF for 15 min, and the procedures are repeated 4 times with refilling excess 10% DMSO-PBS solution. The final product is re-suspended in PBS, adjusting the final volume. The produced Ce6-conjugated antibody is stored at 4° C. Then, the mixture is injected in a patient and allowed to circulate and bind to the disease causing agent. Next, the patient is connected to an extracorporeal tube and a pump and the patient's blood is flowed through the tube into the inlet of the cleansing chamber. With a light source illuminating the chamber, the blood flows through the chamber and the photosensitizer bound to the disease causing agent reacts with a reactive oxygen species, killing the disease causing agent.

According to an embodiment of the present invention, the inner surface of the cleansing device (such as a tube) is bound to a coating material (such as an antibody), wherein the coating material is bound by an intermediate molecule to the inner surface of the cleansing device. In a specific embodiment, the intermediate molecule contains a succinimidyl ester and a carbon chain and maleimidyl ester. The coating material is bound to the intermediate molecule. In some embodiments, the intermediate molecule is a spacer molecule or a zero-length crosslinking agent or any other suitable crosslinking agent.

According to an embodiment of the present invention, light exposure (such as UV 400 nm and 200 nm wavelengths) of the cleansing chamber (such as an extracorporeal tube) is used to kill disease causing agent. Due to its germicidal effects, UV light has been widely used to kill bacteria and viruses. UV irradiation have been suggested and used in surgical wound disinfection and has seen high success in eliminating bacteria. In this disclosure, UV light is used to eliminate blood-borne pathogens as a patient's blood passes through a thin tube before being returned to such patient. This method may be particularly useful for conditions such as sepsis.

According to an embodiment of the present invention, these three individual techniques are combined in different configurations of two and three to remove and kill a disease causing agent. Some combinations are: PDT-Capturing, UV-Capturing, and PDT-UV-Capturing. In some embodiments, the cleansing chambers are connected in a parallel connection. The cleansing chamber for PDT, UV, or both may be illuminated with NIR LED and UV light sources.

According to an embodiment of the present invention, the apparatus is composed of a peristaltic pump and a light source. In the preferred embodiment, a tube passes through a peristaltic pump to maintain the constant flow of blood samples in the tube. The cleansing chamber (for example a tube or several tubes together) is inserted into an illumination chamber, which in some embodiments has cube shape and mirror walls in an inner surface to maximize the light and reflect it from all sides. The temperature inside the illumination chamber is controlled to moderate the heat generated by the light source, such as a 660 nm LED lamp or a UV lamp, in order not to reach temperatures that may damage the blood cells. The output of the cleansing chamber is connected to a tube that returns the blood to a patient. Alternatively, this apparatus may be used to cleanse blood for transfusion or for other purposes. In some embodiments, the initial flow rate is 50 mL/min, with a preferred range of between 30 mL/min and 100 mL/min, and the flow rate through the cleansing chamber is 0.5 mL/min. In a particular embodiment, the tube connected to the blood source (i.e. patient or blood container) is at a high flow rate and the flow rate through the cleansing device is slower. This is achieved by increasing the cross sectional area of the inlet of the cleansing device. As an illustrative example, a tube of 1 mm diameter is connected to a splitter with 100 tubes of 1 mm diameter dropping the flow rate by 100 times.

According to an embodiment of the present invention, the cleansing chambers are unmodified PDMS tubes. In the preferred embodiment, the middle part of the tubes is inserted into the illumination chamber, which is made of mirrors to reflect the light in all directions. In another embodiment, the light source may generate lights of different wavelengths.

In some embodiments, techniques are combined and a surface functionalized tube with an antibody (coating material) can be an effective cleansing chamber for disease causing agents such as blood-borne pathogens.

The utilization of a transparent and thin (inner thickness less than 1 mm) cleansing chamber makes photodynamic therapy (PDT) in blood possible. PDT is based on the activation of photosensitizers by light. The dominant presence of hemoglobin in blood (a strong light absorber) blocks the majority of light necessary to achieve effective PDT. PDT has been used to a limited extent on surface applications where tissue penetration by light is not required (e.g. skin cancer, lung, head, neck cancer, and some dental conditions). In some embodiments, the blood circulates through a thin transparent tube (1.02 mm internal diameter (ID)), and light is illuminated at 360 degree angles by a mirrored chamber. In addition, a near IR photosensitizer, such as Ce6, for example, with an excitation wavelength of 660 nm, can be used to minimize light absorption by hemoglobin.

Thinner cleansing chambers (such as tubes) are associated with higher PDT efficiencies. PDT's efficacy is based upon oxidative damage by locally induced reactive oxygen species. PDT can treat antibiotic-resistant microorganisms, such as MRSA. For the same reason, the photosensitizer is selectively delivered by conjugating with an antibody to target organisms to prevent collateral damage to other blood components. In order to allow sufficient binding between bacteria and Ce6-antibody conjugates, blood samples are circulated for certain given time, and PDT was subsequently performed. Non-specific damage to cells by the reactive oxygen species' (ROS) convection in the blood stream is highly unlikely. Furthermore, PDT is extremely selective to targeted cells.

Using thin transparent cleansing devices enables germicidal light to be used. Germicidal light is defined as light of certain wavelength able to kill bacteria, virus, and microorganisms. Germicidal light having a wavelength between 100 nm and 450 nm is preferred for the disinfection of blood via a light source. In some embodiments, the light source generates short wavelength UV rays (UVC, 207 nm) that selectively kills bacteria with negligible damage to mammalian cells. In some embodiments, the light source generates light that has a wavelength of 200 nm far-UVC. In some embodiments, the light has a wavelength of 207 nm. In some embodiments, the light wavelength is from 400 nm to 100 nm. In some embodiments, the light wavelength is from 290 nm to 100 nm. In some embodiments, the light wavelength ranges between 290 nm to 100 nm. In some embodiments, the light specific has a wavelength between 290 nm to 100 nm. In some embodiments, the light is filtered to allow only the desired wavelength to illuminate the tube. In some embodiments, the filter removes higher-wavelength components. In some embodiments, a low-pass filter with a high-pass filter is used. In some embodiments, a band-pass filter is used to remove wavelengths below and above certain values. In some embodiments, a filter is used to remove wavelengths below 190 nm and above 210 nm. In some embodiments, a filter is used to remove wavelengths below 200 nm and above 210 nm. In some embodiments, a filter is used to remove wavelengths below 100 nm and above 210 nm. In some embodiments, blue light therapy is used. In some embodiments, light has a center wavelength at 415 nm. In some embodiments, light has a center wavelength at 405 nm. In some embodiments, embodiment light has a center wavelength at 405 nm. In some embodiments, light has a center wavelength at 400 nm. In some embodiments, the wavelength of the light is between 395 nm and 445 nm. In some embodiments, the wavelength of the light is higher than 395 nm. In some embodiments, the wavelength of the light is between 375 nm and 465 nm. In some embodiments, the wavelength of the light is between 380 nm and 495 nm. In some embodiments, the wavelength of the light is between 380 nm and 450 nm. In some embodiments, a combination of the above wavelengths is used, for example a light with center wavelength at 207 nm and a light with center wavelength at 415 nm are use simultaneously in the illumination chamber.

According to an embodiment of the present invention, two or three techniques are combined. In some embodiments, in order to enhance efficiency, an additional binding agent (antibody or binding molecule) is introduced so that capturing and PDT use different antibodies to bind to the same bacteria. In some embodiments, the blood flows through multiple tubes each one coated with different coating materials targeting different disease causing agents. In some embodiments, the photosensitizer-antibody conjugates have different antibodies to target different bacteria. In some embodiments, the antibodies or adhesion molecules used for the conjugates and the tubes are all different. In some embodiments, the tubes are removed and placed in culture media, the bacteria are allowed to grow and then fluorescently tagged or otherwise treated to determine the type of bacteria or disease in the blood.

In clinical situations, success or failure of blood-borne infection treatments depends on the timing of the intervention. The required time to isolate responsible microorganisms and to apply appropriate antibiotics often becomes a tremendous challenge for patients with sepsis. The apparatus and methods provided by this invention provide patients with more time to significantly slow down the progress of bacterial growth or potentially stop it. In some embodiments, throughput is increased with the use of multiple tubes in parallel.

According to an embodiment of the present invention, a coating material, such as adhesion molecules (e.g. antibodies), is used to target specific pathogens. In the preferred embodiment, the cleansing chamber and the photosensitizer-antibody conjugates are easily prepared with a specific antibody. In some embodiments, coating materials that target a large group of disease causing agents is used without the need to first identify the disease causing agents. These general purpose molecules are used to coat the cleansing chamber and conjugate to the photosensitizer. In some embodiments, coating materials, such as antibodies or molecules targeting alpha gal (a carbohydrate found in the cell membrane of most organisms, but not in human cells), is used as a target.

According to an embodiment of the present invention, the cleansing chamber is coated with coating materials that include, but are not limited to, pathogen killing agents, that directly kill pathogens. As an illustrative example, agents that inhibit pathogen cell wall biosyntheses, such as beta-lactam antibiotics, or even stronger agents, are employed and coated on the tube. Given that these agents are not taken directly by the patient, but instead reside on an extracorporeal tube, toxicity to the patient is reduced. In some embodiments, the apparatus and method are used to remove pathogens, particles, disease causing organisms, disease causing molecules, toxins, and access molecules that cause disease. Variations of this invention may be used to disinfect and clean contaminated areas and objects. In some embodiments, the apparatus and method are used following a screening procedure to determine the cause of an illness. In some embodiments, the apparatus is used also for diagnostics. For example, the captured organisms are collected and then tagged with die to determine the type of infection.

According to an embodiment of the present invention, the apparatus and methods described by this invention can be specialized for treating a single bacterium, such as MRSA, which is a major problem in hospital infection. The antibiotic free and non-specific nature of the therapy mechanism enables effective treatment on microorganisms regardless of antibiotic resistance. In some embodiments, the cleansing chamber is used as a enrichment device for target organisms. By circulating patient's blood through a series of capturing tubes with coating materials (such as specific antibodies or other targeting molecules), microorganisms distributed in the entire body in very low concentration can be rapidly concentrated in each tubes without necessity for further isolation steps. This significantly reduces the time required for sepsis diagnosis. This invention may be used to clear the blood from gram negative and positive bacteria, parasites, fungi, other unwanted microorganisms, harmful microorganisms, particles, microparticles, nanoparticles, and other disease causing agents and deleterious molecules as described previously. Embodiments of this invention may be used during surgery, for post-surgery recovery and infection control, pre-surgery processes, and for therapeutic applications. Embodiments of this invention can be configured to work in the field, at a hospital or similar medical facility, or in a patient's home.

According to an embodiment of the present invention, any photosensitizer that generates reactive oxygen species, such as singlet oxygen and super oxide, can be used. In a preferred embodiment, suitable photosensitizers include those that do not require conjugation to a binding agent (such as antibody or peptide or adhesion molecule). In other words, embodiments of the present invention may be used with photosensitizers that can directly bind to the disease causing agent (such as a pathogen) without a binding agent. Furthermore, reactive oxygen species (ROS) are oxygen containing chemically reactive molecules.

According to a preferred embodiment of the present invention, the blood flow rate is 0.5 ml/min. In other embodiments, the blood flow rate is any suitable value between 0.01 and 3000 ml/min and can be adjusted according to the specific application or treatment. In some embodiments, the blood flows from the patient into a tube, which is then split into tubes that pass through the illumination chamber (where near IR light, UV light, or any combination thereof can illuminate the tubes) and then the tubes connect to other tubes that are coated with pathogen capturing or killing molecules. The blood then is returned to the patient. In some embodiments, the smaller internal diameter tubes have smaller flow rates. In some embodiments, the larger internal diameter tube has a diameter of 10 mm and the smaller internal diameter tubes have internal diameters of 1 mm. In some embodiments, the flow rate through the first tube connected to the patient is 100 ml/min, with the second tubes (ranging in number from 1 to 400 tubes) have a flow rate of 0.5 ml/min and are smaller in diameter (e.g. 1 mm in diameter). In some embodiments, the second tubes are subjected to the illumination (by IR light, UV light, or any combination thereof). In some embodiments, the second tubes are connected to third tubes that are coated with pathogen capturing or killing molecules.

According to an embodiment of the present invention, blood flows through a tube with a diameter of 1 mm at a flow rate of 50 mL/min. In a preferred embodiment, the tube is connected to a multi-connector junction that is further connected to a multiport manifold with a cleansing device that is made of 100 tubes, each of those tubes being 1 mm in diameter about 1 meter long. In the preferred embodiment, the blood flows through the 100 tubes at about 0.5 mL/min flow and a light source illuminates the 100 tubes with germicidal wavelength. In an alternate preferred embodiment, the light source illuminates the 100 tubes with light of certain wavelength to activate the photosensitizer (for example NIR light). In some embodiments, both light sources are included. In the preferred embodiment, the 100 tubes are connected via another connector to another set 100 tubes of the same size and length that are also pre-coated with coating material. In the preferred embodiment, the coating material is an adhesion molecule or a killing agent. In some embodiments, the apparatus, via a second connector, is connected to a third set of 100 tubes that are of the same size and length as those tubes in the first two sets. The third set of tubes is coated in an additional coating material. In some embodiments, additional groups of 100 tubes are connected. Following the cleansing process, the last set of 100 tubes is connected to a connector that contains only one outlet tube on the other side. In preferred embodiment, the outlet tube is connected to a syringe or similar component that returns the blood into the patient or a container with cleansed blood. The number of tubes, their dimensions, and the flow rates are illustrative in nature, and not to be construed as limiting.

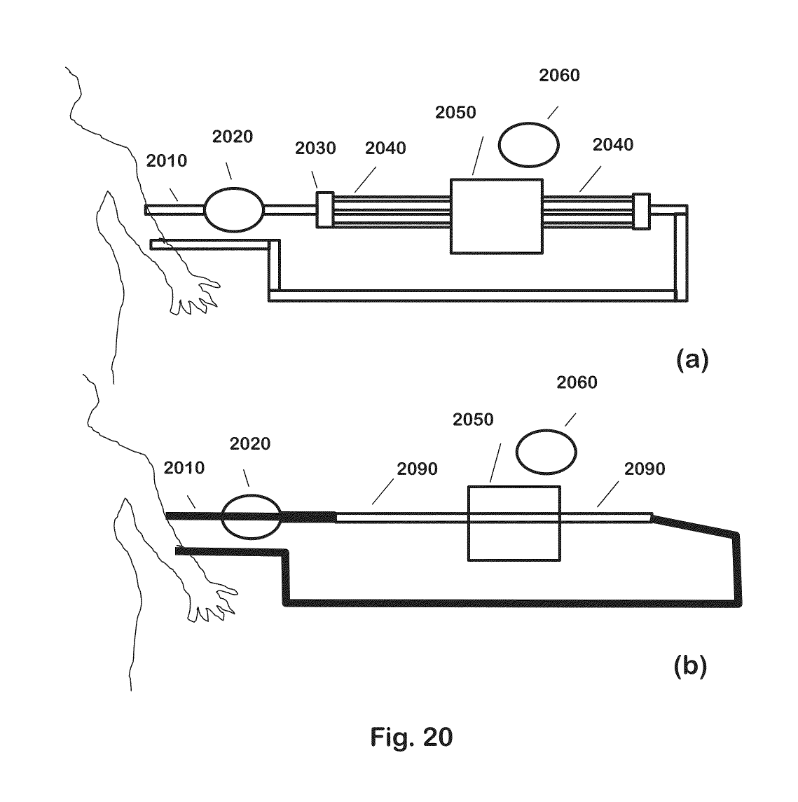

Turning now to FIG. 20(a), a conceptual illustration of a blood cleansing apparatus, in accordance with an embodiment of the present invention. The apparatus begins with a large diameter tube (2010) that carries blood. In a preferred embodiment, the blood is pumped by a pump (2020). A tube splitter (2030) connects the first tube to many tubes (2040), thereby reducing the flow rate. In a preferred embodiment, these tubes (2040) are coated with pathogen capturing or killing molecules or both. The tubes (2040) go through an illumination chamber (2050) where the tubes are illuminated from light generated by a light source such as a light lamp or LED or LASER (2060). In some embodiments more than one light source is included to generate light in a variety of wavelengths. As an illustrative example, one light source provides light in the violet or near ultraviolet spectrum and another light source provides light in the near infrared spectrum. In some embodiments, the cleansing chamber is cooled or placed inside another chamber with lower temperature, for instance at a temperature of 4 Celsius. In some embodiments, only the coated section (or part) a tube with coating material of the cleansing chamber is cooled.

Turning now to FIG. 20(b), a conceptual illustration of a blood cleansing apparatus, in accordance with an embodiment of the present invention. The apparatus begins with a tube (2010) that carries blood that is pumped by a pump (2020). A tube connector connects the first tube (2010) to another tube (2090) coated with pathogen capturing or killing molecules or both. In some embodiments, the second tube (2090) goes through an illumination chamber (2050) where it is illuminated from light generated by a light source such as a light lamp or LED or LASER (2060). In some embodiments more, than one light source is included to generate light in a variety of wavelengths. For instance, one light source provides light in the violet or near ultraviolet spectrum and another light source provides light in the near infrared spectrum. In some embodiments, the tube coated with the coating material (otherwise defined as the cleansing chamber) is cooled or placed inside another chamber with lower temperature, for instance at a temperature of 4 Celsius. In some embodiments, only the section (or part) of the cleansing chamber that includes a coating is cooled.

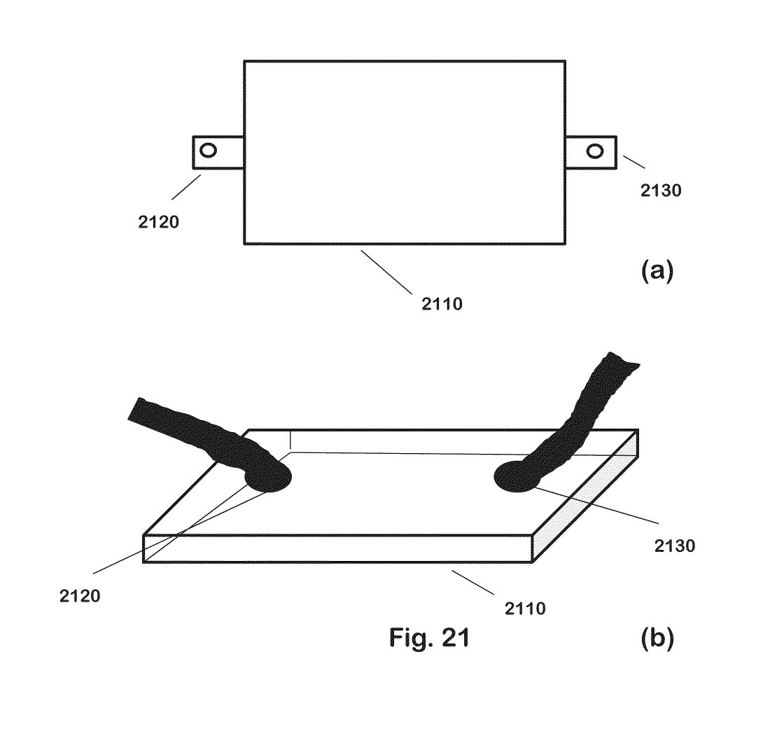

Turning now to FIG. 21, a conceptual illustration of a cleansing chamber, in accordance with an embodiment of the present invention. In a preferred embodiment, the cleansing chamber (2110) has an inlet (2120) and an outlet (2130) for tube connection. In the preferred embodiment, the chamber has a thickness that is less than or equal to 1 mm. Suitable thicknesses for the chamber include, but are not limited to 0.1 mm, 0.5 mm, and 1 mm. In the preferred embodiment, the cleansing chamber is transparent to light.

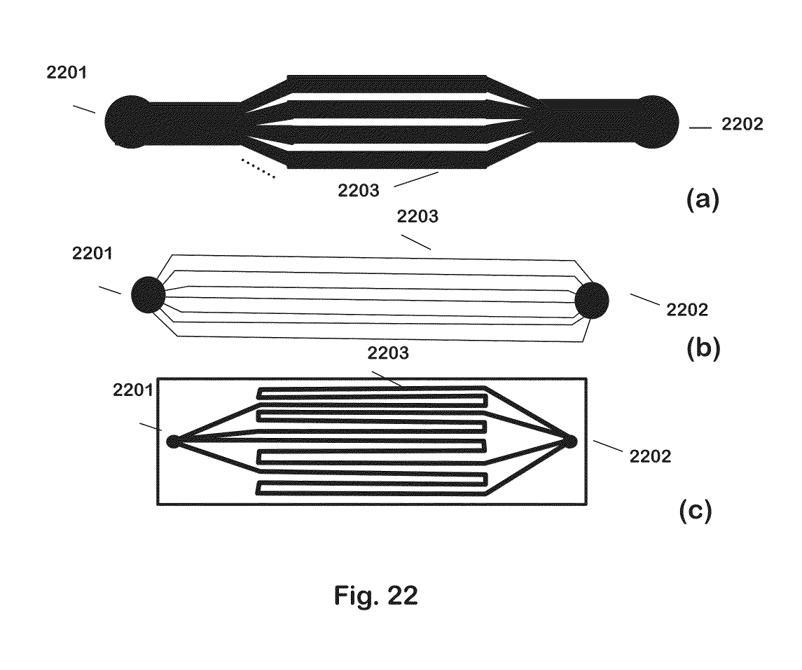

Turning now to FIGS. 22(a)-(c), various conceptual illustrations of a cleansing chamber, in accordance with an embodiment of the present invention. In a preferred embodiment, the cleansing chamber has an inlet (2201) and outlet (2202). In the preferred embodiment the inlet (2201) and outlet (2202) are designed to fit and attach to a tube with multiple channels having the same cross sectional area (2203), for example each channel is 0.5 mm thick, 1 mm wide, 1 meter long. In some embodiments, a cleansing chamber is a plate with inlet (2201), an outlet (2202), and multiple channels (2203). In FIG. 22(c), a cleansing chamber with channels (2203) arranged in a meandering layout like structure is shown. In some embodiments, the plate is 300 mm×300 mm, while in others it is 480 mm×480 mm. In some embodiments, the channels (2203) are transparent to light and rest on a reflective surface such as a thin metal film like gold or silver. In some embodiments, the substrate is a silicon substrate or glass substrate with a reflective layer such as gold or silver for reflection of light on top, with the inlet, outlet, and channels resting on top of the reflective layer.

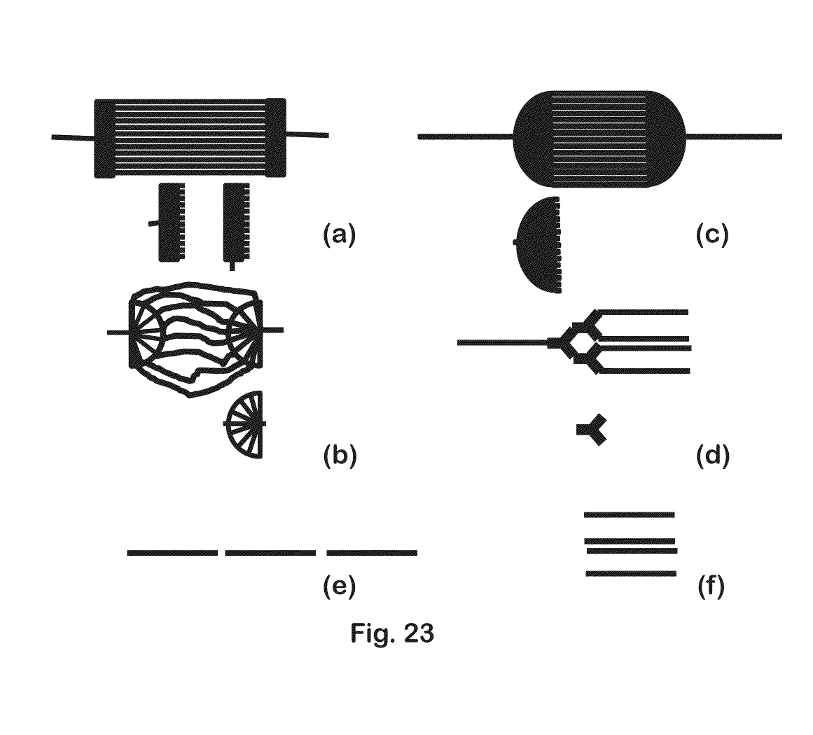

Turning now to FIG. 23 various tube connectors, in accordance with an embodiment of the present invention. In a preferred embodiment, the cleansing device includes a tube connector connecting the first tube to one or more second tubes. In some embodiments, the tube is a medical transparent tube. In some embodiments, a medical extension tube with multiple connectors can be used. In some embodiments, a tube splitter or connector or manifold is used. In some embodiments, as shown in (a), (b), and (c), the splitter or manifold connects one tube to multiple tubes. In some embodiments, the splitter splits the first tube into two then the resulting two tubes are split into four using another splitter. In some embodiments, as shown in (b) and (c) the tube manifold is semicircular. In some embodiments, as shown in FIG. 23 (e), the tubes are connected in series and each tube has a different coating material. In the preferred embodiment, different coating materials serve to capture or kill different disease causing agents. In some embodiments, as shown in FIG. 23 (f), the tubes are connected in parallel and each tube has a different coating material. In some embodiments each tube may be analyzed to determine the type or kind of disease causing agent. For instance, a die may be used to indicate the presence of a disease causing agent like a bacterium. If the bacterium is present, then a florescent color would be present.

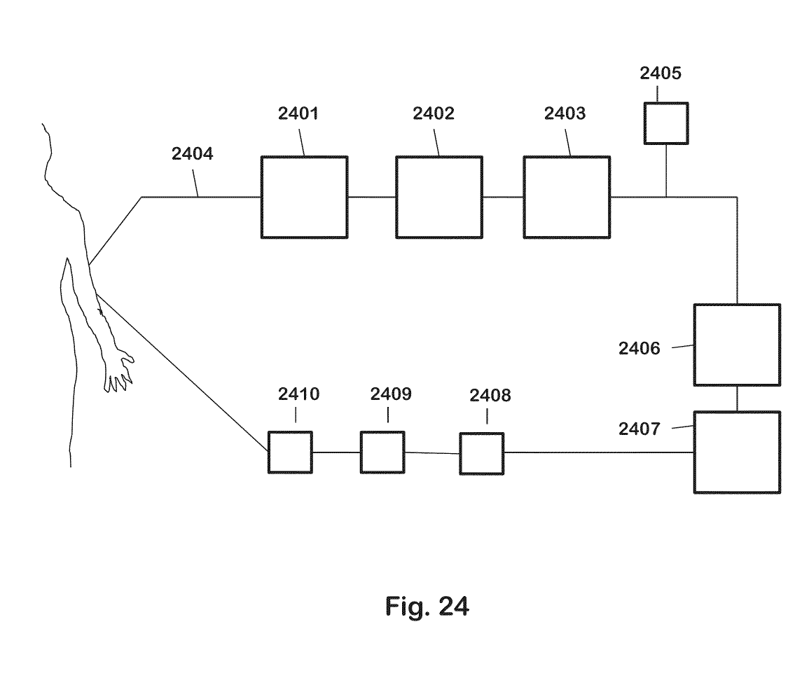

Turning now to FIG. 24, a conceptual illustration of the apparatus disclosed configured as a dialysis-like apparatus or part of a dialysis machine. In a preferred embodiment, blood flows through a tube (2404) from patient to an arterial pressure monitor (2401), then into a pump (2402). A pump with anticoagulant, such as heparin (2403), is connected to ensure there is no coagulation and to prevent clotting. In preferred embodiment, a saline solution (2405) is also included. In the preferred embodiment, the tube (2404) then connects to a dialyser (2406). At the top of the dialyser, fresh dialysate is pumped in and at the bottom used dialysate is removed (not shown). The dialyser (2406) removes toxins including microbial toxins and toxins produced by micro-organisms. The blood then flows through a tube into a cleansing device (2407). In some embodiments, said cleansing device (2407) is a tube coated with coating material. In some embodiments, the tube is exposed to light of specific wavelength as the ones described earlier. In some embodiments, the apparatus includes a filter (2408) that removes items larger than several microns, for example objects larger than 40 microns in diameter. In some embodiments, a venous pressure monitor (2409) is included. In some embodiments, an air trap and air detector (2410) is also included. Finally, the blood is recirculated back to the patient. In some embodiments, the apparatus is part of a dialysis machine.

According to an embodiment of the present invention, the blood cleansing apparatus is used to clean a blood source. In a first preferred embodiment, the blood source is a patient receiving treatment. In a second preferred embodiment, the blood source is a blood reserve that that requires cleansing before it can be used. As an illustrative example, a blood reserve could be blood donated to a blood bank. Likewise, a blood source could be a blood donor that is donating blood to a blood bank, hospital, or similar healthcare provider. In a third preferred embodiment, a blood source could be the source of blood being used for a blood transfusion or similar procedure. One of ordinary skill in the art would appreciate that a blood source could be any source of blood that requires cleansing before being returned to or use by a patient.

The methods and embodiments of the present invention are adaptable to any adhesion molecule and can be used to reduce infectious particle load to minimal levels or at levels where conventional medication and the body's own immune system can fight the infection. This disclosure is particularly useful for individuals experiencing immunosuppression or young children for whom antibiotics and antifungal medication can be highly toxic.

While the invention has been thus described with reference to the embodiments, it will be readily understood by those skilled in the art that equivalents may be substituted for the various elements and modifications made without departing from the spirit and scope of the invention. It is to be understood that all technical and scientific terms used in the present invention have the same meaning as commonly understood by one of ordinary skill in the art to which this invention belongs. Accordingly, the drawings and descriptions are to be regarded as illustrative in nature and not restrictive.

1. An apparatus for removing disease causing agent from blood, said apparatus comprising:
an inlet tube for flowing blood from a blood source;
a pump connected to the tube;
one or more cleansing chambers connected to the tube, wherein each of the cleansing chambers comprises an inlet, through which the blood flows in to the cleansing chamber, an outlet, through which the blood flows out of the cleansing chamber, an outlet, and an inner portion coated with a coating material; and
an outlet tube connected to the outlet of the cleansing chamber which returns the blood to the blood source.

2. The apparatus of claim 1, wherein each of the cleansing chambers is one or more cleansing chambers selected from a group of cleansing chambers comprising tube, parallelepiped, rectangular parallelepiped, and a cylinder.

3. The apparatus of claim 1, wherein the coating material is one or more coating materials selected from a group of coating material comprising antibodies, adhesion molecules, and pathogen killing molecules.

4. The apparatus of claim 1, further comprising one or more light sources each of which illuminate at least one of the one or more cleansing chambers.

5. The apparatus of claim 4, wherein the light sources generate light with one or more of wavelengths selected from a group of wavelengths comprising a wavelength centered at 207 nm, a wavelength centered at 415 nm, a wavelength centered at 400 nm, a wavelength centered at 405 nm, a wavelength centered at 200 nm, a wavelength between 100 nm and 210 nm, a wavelength between 100 nm and 400 nm, a wavelength between 380 nm and 450 nm, 660 nm, a wavelength between 650 nm and 700 nm, a wavelength between 700 nm and 900 nm, a wavelength longer than 400 nm, and a wavelength that activates a photosensitizer.

6. The apparatus of claim 1, wherein the blood source is a patient receiving treatment.

7. The apparatus of claim 1, further comprising a port connected to the outlet tube, wherein the port is used to introduce a photosensitizer to the blood source.

8. An apparatus for removing disease causing agent from blood of a patient, said apparatus comprising:
an inlet tube for flowing blood from a blood source;
a pump connected to the tube;
one or more cleansing chambers connected to the tube, wherein each of the cleansing chambers comprises an inlet, through which the blood flows in to the cleansing chamber, an outlet, through which the blood flows out of the cleansing chamber, and an outlet;
one or more light sources each of which illuminate at least one of the one or more cleansing chambers; and
an outlet tube connected to the outlet of the cleansing chamber which returns the blood to the blood source.

9. The apparatus of claim 8, wherein the light sources generate light with one or more of wavelengths selected from a group of wavelengths comprising a wavelength centered at 207 nm, a wavelength centered at 415 nm, a wavelength centered at 400 nm, a wavelength centered at 405 nm, a wavelength centered at 200 nm, a wavelength between 100 nm and 210 nm, a wavelength between 100 nm and 400 nm, a wavelength between 380 nm and 450 nm, 660 nm, a wavelength between 650 nm and 700 nm, a wavelength between 700 nm and 900 nm, a wavelength longer than 400 nm, and a wavelength that activates a photosensitizer

10. The apparatus of claim 8, wherein each of the cleansing chambers is one or more cleansing chambers selected from a group of cleansing chambers comprising tube, parallelepiped, rectangular parallelepiped, and a cylinder.

11. The apparatus of claim 8, further comprising a coating material on an inner part of one or more of the cleansing chambers, wherein the coating material is one or more coating materials selected from a group of coating material comprising antibodies, adhesion molecules, and pathogen killing molecules.

12. The apparatus of claim 8, wherein the blood source is a patient receiving treatment.

13. The apparatus of claim 8, further comprising a port connected to the outlet tube, wherein the port is used to introduce a photosensitizer to the blood source.

14. A method for removing disease causing agent from blood, said method comprising the steps of:
flowing blood from a patient through a tube to a first cleansing chamber;
illuminating the blood with light from a light source that generates light with wavelength one or more of wavelengths selected from a group of wavelengths comprising a wavelength centered at 207 nm, a wavelength centered at 415 nm, a wavelength centered at 400 nm, a wavelength centered at 405 nm, a wavelength centered at 200 nm, a wavelength between 100 nm and 210 nm, a wavelength between 100 nm and 400 nm, a wavelength between 380 nm and 450 nm, 660 nm, a wavelength between 650 nm and 700 nm, a wavelength between 700 nm and 900 nm, a wavelength longer than 400 nm, and a wavelength that activates a photosensitizer;
flowing blood through a second cleansing chamber configured with a coating material on an inner part of the second cleansing chamber, wherein the coating material is one or more coating materials selected from a group of coating material comprising antibodies, adhesion molecules, and pathogen killing molecules; and
flowing blood out of the second cleansing chamber into a tube.

15. The method of claim 14, further comprising the steps of:
injecting a photosensitizer into the patient, wherein the photosensitizer attaches to the disease causing agent; and
illuminating the first cleansing chamber with light from the light source to activate the photosensitizer, wherein the activation of the photosensitizer generates reactive oxygen species that cause cell death to the disease causing agent upon contact with the reactive oxygen species.

16. The method of claim 14, wherein the second cleansing chamber is configured with one or more additional binding agents to capture said disease causing agent.
